(19) United States
(12) Patent Application Publication
(10) Pub. No.: US 2026/0265393 A1
(43) Pub. Date: Sep. 10, 2026

(54) ANTI-CD27 MONOCLONAL ANTIBODY AND USE THEREOF

(71) Applicant: SHANGHAI CELGEN BIO-PHARMACEUTICAL CO., LTD., Shanghai (CN)

(72) Inventors: Zeling CAI, Shanghai (CN); Yi CHEN, Shanghai (CN)

(21) Appl. No.: 19/165,731

(22) PCT Filed: Oct. 13, 2023
(86) PCT No.: PCT/CN2023/124588

§ 371 Date: Mar. 9, 2026

(30) Foreign Application Priority Data
Mar. 16, 2023 (CN) 202310256384.2

(51) Int. Cl. C07K 16/28; A61K 35/17; A61K 38/00; A61K 40/11; A61K 40/31; A61K 40/42; A61K 47/68; C07K 14/705; C12N 5/078; C12N 15/11

(52) U.S. Cl. CPC C07K 16/2878; A61K 35/17; A61K 38/00; A61K 40/11; A61K 40/31; A61K 40/4215; A61K 47/6849; C07K 14/70503; C12N 5/0634; C12N 15/11; C07K 2317/24; C07K 2317/51; C07K 2317/52; C07K 2317/565; C07K 2317/622; C07K 2317/71; C07K 2317/92; C12N 2510/00

(57) ABSTRACT

The present invention provides an anti-CD27 monoclonal antibody and a preparation thereof. Particularly, the present invention provides a novel anti-CD27 antibody. The antibody of the present invention can bind to an antigen with high specificity, has high affinity and high biological activity, and can specifically bind to human CD27 antigen molecules. Therefore, the antibody is expected to be used for producing drugs capable of efficiently carrying out immunotherapy in vitro and in vivo.

### TECHNICAL FIELD

The present invention relates to the field of medicine, and in particular to an anti-CD27 humanized monoclonal antibody and a preparation thereof.

### BACKGROUND

CD27, a member of the TNFR superfamily, is a type I transmembrane glycoprotein with a molecular weight of approximately 55 kDa, typically present as a disulfide-linked homodimer. CD27 is expressed as a surface antigen on most T cells, natural killer cells, antibody-secreting plasma cells, and memory B cells. CD70, as a ligand for CD27, interacts with CD27 and recruits intracellular TRAF proteins to intracellular domain of CD27, thereby activating downstream signaling. Normally, after binding to CD70, intracellular CD27 activates the NF-κB and JNK signaling pathways by ligating the TNF receptor-associated molecules TRAF2 and TRAF5, promoting T cell proliferation and the secretion of corresponding cytokines. Studies have shown that CD27-CD70-mediated costimulation plays an important role in T cell growth, differentiation, and survival. It also promotes B cell proliferation, differentiation into plasma cells, immunoglobulin production, and induces NK cell activation.

CD27 is considered a promising target for cancer immunotherapy. Currently, several CD27 antibodies are in clinical trials, including Varlilumab, a CD27 monoclonal antibody developed by Celldex Therapeutics, which has entered Phase II clinical trials. WO2011/130434 discloses this agonistic anti-human CD27 antibody, which activates CD27 upon cross-linking. In vivo experiments have shown that Varlilumab enhances immune cell proliferation and cytokine release in mice, strengthens the immune response to vaccines, and enhances anti-tumor activity in various tumor models. Clinical trials are ongoing for Varlilumab in combination with anti-PD-1 and anti-PD-L1 monoclonal antibodies, which has been shown to be well tolerated, offering new therapeutic prospects for patients with tumors that are typically resistant to PD-1 inhibitor monotherapy. Aduro BioTech, based on its single B cell screening monoclonal antibody technology platform, has developed the anti-CD27 agonistic monoclonal antibody MK-5890, which is currently in Phase II clinical trials. The disclosed patent WO2012/004367 describes the first anti-human agonistic antibody (named hCD27.15), which can activate CD27-mediated co-stimulation of the NF-κB immune response. Separately, Bristol-Myers Squibb (BMS) disclosed patent WO2019195452A1, which discloses a non-ligand-blocking CD27 antibody that can activate T cells and exhibit anti-tumor activity in mouse tumor models.

In summary, CD27 is a promising new immunotherapy target. However, there are currently no CD27 monoclonal antibody drugs have been approved for marketing in China or abroad, so there is a need to develop CD27 monoclonal antibodies with better clinical efficacy.

### SUMMARY OF THE INVENTION

The purpose of the present invention is to provide a CD27 antibody with high affinity and high biological activity and uses thereof.

In the first aspect of the present invention, it provides a heavy chain variable region of an antibody, wherein the heavy chain variable region has three complementarity determining regions (CDRs) selected from the group consisting of:
(a) CDR1, CDR2 and CDR3 shown in SEQ ID No: 2, 3 and 4;
(b) CDR1, CDR2 and CDR3 shown in SEQ ID No: 2, 25 and 4;
(c) CDR1, CDR2 and CDR3 shown in SEQ ID No: 2, 26 and 4;
(d) CDR1, CDR2 and CDR3 shown in SEQ ID No: 10, 11 and 12.

In another preferred embodiment, the heavy chain variable region comprises the following three complementarity determining regions (CDRs):
(1) complementarity determining region CDR1, wherein the amino acid sequence of the complementarity determining region CDR1 is shown in SEQ ID No: 2;
(2) complementarity determining region CDR2, wherein the amino acid sequence of the complementarity determining region CDR2 is shown in SEQ ID No: 3, 25 or 26; and
(3) complementarity determining region CDR3, the amino acid sequence of the complementary determining region CDR3 is shown in SEQ ID No: 4.

In another preferred embodiment, the heavy chain variable region comprises four framework regions FR, and the four framework regions FR are separated by the above-mentioned CDR1, CDR2 and CDR3.

In another preferred embodiment, the heavy chain variable region has an amino acid sequence shown in SEQ ID No: 1, 17, 18, 19, or 20.

In another preferred embodiment, the heavy chain variable region has the amino acid sequence shown in SEQ ID No: 9.

In the second aspect of the present invention, it provides an antibody heavy chain, wherein the antibody heavy chain has the heavy chain variable region of the antibody of the first aspect of the present invention.

In another preferred embodiment, the constant region of the heavy chain is of human origin.

In another preferred embodiment, the constant region of the heavy chain is the heavy chain constant region of IgG1, IgG2 or IgG4.

In another preferred embodiment, the heavy chain constant region is a wild-type Fc or a mutant Fc.

In another preferred embodiment, the mutant Fc causes the loss or substantial loss of ADCC effect of the antibody.

In another preferred embodiment, the mutant Fc has L234A and L235A mutations.

In another preferred embodiment, the heavy chain sequence is shown in SEQ ID NO: 27, and the constant region of the heavy chain is a wild-type Fc.

In another preferred embodiment, the heavy chain sequence is shown in SEQ ID NO: 28, and the constant region of the heavy chain is a mutant Fc, which is the Fc of IgG1 having L234A and L235A mutations.

In the third aspect of the present invention, it provides a light chain variable region of an antibody, wherein the light chain variable region has three complementarity determining regions (L-CDRs) with sequences shown in SEQ ID No: 6, 7, and 8;
or the light chain variable region has three complementarity determining regions L-CDRs with sequences shown in SEQ ID No: 14, 15, and 16.

In another preferred embodiment, the light chain variable region comprises the following three complementarity determining regions L-CDR:
(1) complementarity determining region L-CDR1, wherein the amino acid sequence of the complementarity determining region L-CDR1 is shown in SEQ ID No: 6;
(2) complementarity determining region L-CDR2, the amino acid sequence of which is shown in SEQ ID No: 7; and
(3) complementarity determining region L-CDR3, the amino acid sequence of the complementarity determining region L-CDR3 is shown in SEQ ID No: 8.

In another preferred embodiment, the light chain variable region comprises four framework regions FR, and the four framework regions FR are separated by the above-mentioned CDR1, CDR2 and CDR3.

In another preferred embodiment, the light chain variable region has an amino acid sequence shown in SEQ ID No: 5, 21, 22, 23 or 24.

In another preferred embodiment, the light chain variable region has the amino acid sequence shown in SEQ ID No: 13.

In the fourth aspect of the present invention, it provides an antibody light chain, wherein the antibody light chain has the light chain variable region of the antibody of the third aspect of the present invention.

In another preferred embodiment, the constant region of the light chain is of human origin.

In another preferred embodiment, the constant region of the light chain is a Kappa or Lambda light chain constant region.

In the fifth aspect of the present invention, it provides an antibody, wherein the antibody has:
(1) the heavy chain variable region of the first aspect of the present invention; and/or
(2) the light chain variable region of the third aspect of the present invention.
or, the antibody has: the heavy chain of the second aspect of the present invention; and/or the light chain of the fourth aspect of the present invention.

In another preferred embodiment, the antibody is an anti-CD27 antibody.

In another preferred embodiment, the antibody further comprises a heavy chain constant region and a light chain constant region.

In another preferred embodiment, the heavy chain constant region of the antibody is a wild-type Fc or a mutant Fc.

In another preferred embodiment, the mutant Fc causes the lost or substantially los of ADCC effect of the antibody.

In another preferred embodiment, the mutant Fc has L234A and L235A mutations.

In another preferred embodiment, the heavy chain sequence of the antibody is shown in SEQ ID NO: 27, and the constant region of the heavy chain is a wild-type Fc.

In another preferred embodiment, the heavy chain sequence of the antibody is as shown in SEQ ID NO: 28, and the constant region of the heavy chain is a mutant Fc, which is the Fc of IgG1 having L234A and L235A mutations.

In another preferred embodiment, the antibody has a heavy chain variable region as shown in SEQ ID No: 1, 17, 18, 19 or 20; and/or a light chain variable region as shown in SEQ ID No: 5, 21, 22, 23 or 24.

In another preferred embodiment, the antibody has a heavy chain variable region as shown in SEQ ID No: 9; and/or a light chain variable region as shown in SEQ ID No: 13.

In another preferred embodiment, the antibody is selected from the following group consisting of:
(Z1) an antibody having a heavy chain variable region shown in SEQ ID No: 1 and a light chain variable region shown in SEQ ID No: 5:
(Z2) an antibody having a heavy chain variable region shown in SEQ ID No: 18 and a light chain variable region shown in SEQ ID No: 22;
(Z3) an antibody having a heavy chain variable region shown in SEQ ID No: 18 and a light chain variable region shown in SEQ ID No: 23;
(Z4) an antibody having a heavy chain variable region shown in SEQ ID No: 18 and a light chain variable region shown in SEQ ID No: 24;
(Z5) an antibody having a heavy chain variable region shown in SEQ ID No: 19 and a light chain variable region shown in SEQ ID No: 24;
(Z6) An antibody having the heavy chain variable region shown in SEQ ID No: 20 and the light chain variable region shown in SEQ ID No: 21.

In another preferred embodiment, the antibody is a humanized antibody, a chimeric antibody, or a murine antibody.

In another preferred embodiment, the antibody specifically binds to CD27.

In another preferred embodiment, the antibody has the function of blocking the binding of CD70 to CD27.

In another preferred embodiment, the antibody has the function of activating T cells and promoting T cell proliferation.

In another preferred embodiment, the antibody is a double-chain antibody or a single-chain antibody.

In another preferred embodiment, the antibody is a monoclonal antibody.

In another preferred embodiment, the antibody is a bispecific antibody or a multispecific antibody.

In another preferred embodiment, the antibody is in the form of a drug conjugate.

In the sixth aspect of the present invention, it provides a recombinant protein, wherein the recombinant protein has:
(i) the heavy chain variable region of the first aspect of the invention, the heavy chain of the second aspect of the invention, the light chain variable region of the third aspect of the invention, the light chain of the fourth aspect of the invention, or the antibody of the fifth aspect of the invention; and
(ii) optionally a tag sequence to facilitate expression and/or purification.

In another preferred embodiment, the tag sequence comprises a 6His tag.

In another preferred embodiment, the recombinant protein (or polypeptide) comprises a fusion protein.

In another preferred embodiment, the recombinant protein is a monomer, a dimer, or a multimer.

In the seventh aspect of the present invention, it provides an antibody formulation, comprising:
(a) the antibody of the fifth aspect of the present invention; and
(b) a carrier or excipient.

In another preferred embodiment, the formulation is a pharmaceutical composition.

In another preferred embodiment, the excipient or carrier is a pharmaceutically acceptable carrier or excipient.

In another preferred embodiment, the carrier comprises: a buffer, sterile water, and optionally a surfactant.

In another preferred embodiment, in the formulation, the concentration of the antibody is 5-100 mg/mL; preferably 10-70 mg/mL, more preferably 20-60 mg/mL.

In another preferred embodiment, the buffer is selected from the group consisting of: PBS buffer system, citric acid buffer system, histidine buffer system, or a combination thereof.

In another preferred embodiment, the pH range of the preparation is 5.0-7.5, preferably 5.5-7.

In another preferred embodiment, the formulation is an injection.

In the eighth aspect of the present invention, it provides a kit, wherein the kit comprises the antibody of the fifth aspect of the present invention and a container for containing the antibody.

In the ninth aspect of the present invention, it provides a CAR construct, wherein the scFv segment of the antigen binding region of the CAR construct is a binding region that specifically binds to CD27, and the scFv has the heavy chain variable region of the first aspect of the present invention and the light chain variable region of the third aspect of the present invention.

In the tenth aspect of the present invention, it provides a recombinant immune cell, which expresses exogenous CAR construct of the ninth aspect of the present invention.

In another preferred embodiment, the immune cell is selected from the group consisting of: NK cell and T cell.

In another preferred embodiment, the immune cell is from human or non-human mammal (such as mouse).

In the eleventh aspect of the present invention, it provides an antibody-drug conjugate, wherein the antibody-drug conjugate comprises:
(a) an antibody portion selected from the group consisting of: the heavy chain variable region of the first aspect of the invention, the heavy chain of the second aspect of the invention, the light chain variable region of the third aspect of the invention, the light chain of the fourth aspect of the invention, or the antibody of the fifth aspect of the invention, or a combination thereof; and
(b) a conjugated moiety conjugated to the antibody portion, wherein the conjugated moiety is selected from the group consisting of: a detectable label, a drug, a toxin, a cytokine, a radionuclide, an enzyme, or a combination thereof.

In another preferred embodiment, the antibody portion and the conjugated moiety are conjugated via a chemical bond or a linker.

In the twelfth aspect of the present invention, it provides a use of an active ingredient, wherein the active ingredient is selected from the group consisting of: the heavy chain variable region of the first aspect of the present invention, the heavy chain of the second aspect of the present invention, the light chain variable region of the third aspect of the present invention, the light chain of the fourth aspect of the present invention, or the antibody of the fifth aspect of the present invention, the recombinant protein of the sixth aspect of the present invention, the immune cell of the tenth aspect of the present invention, the antibody-drug conjugate of the eleventh aspect of the present invention, or a combination thereof, wherein the active ingredient is used for
(a) preparing a detection reagent or kit;
(b) preparing a medicine or preparation for preventing and/or treating a CD27-related disease; and/or
(c) preparing a medicine or preparation for preventing and/or treating a cancer or tumor.

In another preferred embodiment, the tumor is selected from the group consisting of: hematological tumors, solid tumors, or a combination thereof.

In another preferred embodiment, the hematological tumor is selected from the group consisting of: acute myeloid leukemia (AML), multiple myeloma (MM), chronic lymphocytic leukemia (CLL), acute lymphocytic leukemia (ALL), diffuse large B-cell lymphoma (DLBCL), Hodgkin's lymphoma, or a combination thereof.

In another preferred embodiment, the solid tumor is selected from the group consisting of: gastric cancer, gastric cancer peritoneal metastasis, liver cancer, leukemia, kidney tumor, lung cancer, small intestine cancer, bone cancer, prostate cancer, colorectal cancer, breast cancer, large intestine cancer, cervical cancer, ovarian cancer, lymphoma, nasopharyngeal carcinoma, adrenal tumor, bladder tumor, non-small cell lung cancer (NSCLC), brain glioma, endometrial cancer, or a combination thereof.

In another preferred embodiment, the medicine or preparation is used to prepare a medicine or preparation for preventing and/or treating a disease associated with CD27 (positive expression).

In another preferred embodiment, the antibody is in the form of an antibody-drug conjugate (ADC).

In another preferred embodiment, the detection reagent or kit is used to diagnose a CD27-related disease.

In another preferred embodiment, the detection reagent or kit is used to detect CD27 protein in a sample.

In another preferred embodiment, the detection reagent is a detection plate.

In the thirteenth aspect of the present invention, it provides a pharmaceutical composition, comprising:
(i) an active ingredient, the active ingredient is selected from the group consisting of: the heavy chain variable region of the first aspect of the invention, the heavy chain of the second aspect of the invention, the light chain variable region of the third aspect of the invention, the light chain of the fourth aspect of the invention, the antibody of the fifth aspect of the invention, the recombinant protein of the sixth aspect of the invention, the immune cell of the tenth aspect of the invention, the antibody-drug conjugate of the eleventh aspect of the invention, or a combination thereof; and
(ii) a pharmaceutically acceptable carrier.

In another preferred embodiment, the pharmaceutical composition further comprises a second anti-tumor active ingredient.

In another preferred embodiment, the second active ingredient is selected from the group consisting of: a cytotoxic drug, toxin, cytokine, enzyme, antibody, or a combination thereof.

In another preferred embodiment, the second active ingredient comprises: an antibody targeting EGFR and an antibody targeting HER2.

In another preferred embodiment, the pharmaceutical composition is a liquid preparation.

In another preferred embodiment, the pharmaceutical composition is an injection.

In another preferred embodiment, the pharmaceutical composition is used to treat a tumor.

In the fourteenth aspect of the present invention, it provides a polynucleotide, wherein the polynucleotide encodes a polypeptide selected from the group consisting of:
(1) the heavy chain variable region of the first aspect of the present invention, the heavy chain of the second aspect of the present invention, the light chain variable region of the third aspect of the present invention, the light chain of the fourth aspect of the present invention, or the antibody of the fifth aspect of the present invention; or
(2) the recombinant protein of the sixth aspect of the present invention; and/or
(3) the CAR construct of the ninth aspect of the present invention.

In the fifteenth aspect of the present invention, it provides a vector, wherein the vector comprises the polynucleotide of the fourteenth aspect of the present invention.

In another preferred embodiment, the vector comprises: a bacterial plasmid, bacteriophage, yeast plasmid, plant cell virus, mammalian cell virus such as adenovirus, retrovirus, or other vectors.

In the sixteenth aspect of the present invention, it provides a genetically engineered host cell, wherein the host cell comprises the vector of the fifteenth aspect of the present invention or the polynucleotide of the fourteenth aspect of the present invention is integrated into its genome.

In the seventeenth aspect of the present invention, it provides a method for detecting CD27 protein in a sample (including diagnostic or non-diagnostic) in vitro, the method comprising the steps of:
(1) contacting the sample with the antibody of the fifth aspect of the present invention in vitro;
(2) detecting whether an antigen-antibody complex is formed, wherein the formation of the complex indicates the presence of CD27 protein in the sample.

In the eighteenth aspect of the present invention, it provides a detection plate, which comprises: a substrate (support plate) and a test strip, wherein the test strip comprises the antibody of the fifth aspect of the present invention or the antibody-drug conjugate of the eleventh aspect of the present invention.

In the nineteenth aspect of the present invention, it provides a kit, comprising:
(1) a first container, wherein the first container contains the antibody of the fifth aspect of the present invention; and/or
(2) a second container containing a secondary antibody against the antibody of the fifth aspect of the present invention;
or, the kit comprises the detection plate of the eighteenth aspect of the present invention.

In the twentieth aspect of the present invention, it provides a method for preparing a recombinant polypeptide, the method comprising:
(a) culturing the host cell of the fourteenth aspect of the present invention under a condition suitable for expression;
(b) isolating the recombinant polypeptide from the culture, wherein the recombinant polypeptide is the antibody of the fifth aspect of the present invention or the recombinant protein of the sixth aspect of the present invention.

In the twenty-first aspect of the present invention, it provides a method for treating a CD27-related disease, such as a cancer, the method comprising administering to a subject in need thereof the antibody of the fifth aspect of the present invention, the antibody-drug conjugate of the antibody, or the CAR-T cell expressing the antibody, or a combination thereof.

In another preferred embodiment, the subject comprises a human and non-human mammal.

In another preferred embodiment, the subject is a human.

It should be understood that within the scope of the present invention, the various technical features of the present invention above and the various technical features specifically described hereinafter (as in the examples) may be combined with each other to constitute a new or preferred technical solution. And it needs not be described one by one, due to space limitations.

### DESCRIPTION OF THE DRAWINGS

FIG. 1 shows SDS-PAGE analysis of the antibodies of the present invention.

FIG. 2 shows the ELISA test results of the antibodies of the present invention.

FIG. 3 shows the binding effect of the antibodies of the present invention to the CD27 protein on the CHO cell membrane.

FIG. 4 shows the binding effect of human-mouse chimeric CD27 antibodies to the CD27 on the surface of human T and B cells detected by FACS.

FIG. 5 shows the inhibitory effect of human-mouse chimeric CD27 antibodies on the binding of CD27 to CD70.

FIG. 6 shows the human-mouse chimeric CD27 monoclonal antibodies binding epitope experiment.

FIG. 7 shows the binding epitope experiment of 13B3 and 1F5.

FIG. 8 shows the effect of anti-CD27 antibodies in promoting the proliferation of CD3+ T cells.

FIG. 9 shows that the anti-CD27 antibodies inhibit the CD27 downstream NFκB signaling pathway by blocking the binding of CD70 on the surface of Raji cells to CD27 on the surface of Jurkat cells.

FIG. 10 shows the ELISA results of the in vitro binding activity of humanized monoclonal CD27 antibodies to the antigen CD27.

FIG. 11 shows the flow cytometry results of the binding of humanized CD27 monoclonal antibodies to CD27 protein on the cell membrane.

FIG. 12 shows the inhibitory effect of humanized CD27 antibodies on the binding of CD27 to CD70.

FIG. 13 shows the promoting effect of humanized antibodies on T cell proliferation.

FIG. 14 shows the changes in the percentage of T and B lymphocytes in the peripheral blood of mice 3 days after the last administration of the antibody of the present invention.

FIG. 14A: CD3+ T lymphocytes; FIG. 14B: CD3+CD4+ T lymphocytes; FIG. 14C: CD3+CD4− T lymphocytes; FIG. 14D: CD19+ B lymphocytes.

FIG. 15 shows the changes in the percentage of T lymphocytes in the peripheral blood of mice 10 days after the last administration of the antibody of the present invention. FIG. 15A: CD3+ T lymphocytes; FIG. 15B: CD3+CD4+ T lymphocytes; FIG. 15C: CD3+CD8+ T lymphocytes.

FIG. 16 shows the changes in the percentage of T lymphocytes in the spleen of mice 10 days after the last administration of the antibody of the present invention. FIG. 16A: CD3+ T lymphocytes; FIG. 16B: CD3+CD4+ T lymphocytes; FIG. 16C: CD3+CD8+ T lymphocytes.

FIG. 17 shows the effects of humanized antibodies alone or in combination with other immune checkpoint inhibitor antibodies on IFNγ production by T cells at the exhaustion stage.

FIG. 18 shows the in vitro binding activities of wild-type and variant CD27 antibodies to CD27.

FIG. 19 shows the inhibitory effects of wild-type and variant CD27 antibodies on the binding of CD27 to CD70 in vitro.

FIG. 20 shows the binding activity of wild-type and variant CD27 antibodies to Fc receptors CD32a and CD64 on the cell surface. FIG. 20A: Binding to CD32a on the cell surface; FIG. 20B: Binding to CD64 on the cell surface.

FIG. 21 shows the changes in T lymphocyte percentages in the peripheral blood of mice 3 days after the last administration of wild-type and variant CD27 antibodies of the present invention. FIG. 21A: CD3+ T lymphocytes; FIG. 21B: CD3+CD4+ T lymphocytes; FIG. 21C: CD3+CD8+ T lymphocytes.

FIG. 22 shows the changes in T lymphocyte percentages in the peripheral blood of mice 10 days after the last administration of wild-type and variant CD27 antibodies of the present invention. FIG. 22A: CD3+ T lymphocytes; FIG. 22B: CD3+CD4+ T lymphocytes; FIG. 22C: CD3+CD8+ T lymphocytes.

### DETAILED DESCRIPTION

Through extensive and deep research, after a large-scale screening, the inventors have obtained multiple murine antibodies with excellent properties, including high affinity for human CD27. Based on these murine antibodies with high affinity and high anti-tumor activity, chimeric antibodies and humanized antibodies were further developed. The antibodies of the present invention effectively bind to human CD27 and exhibit excellent activity, making them suitable for use as monoclonal antibody drugs for targeted therapy. The present invention has been completed on this basis.

### Term

In order to more easily understand the present disclosure, some terms are first defined. As used in this application, unless otherwise expressly provided herein, each of the following terms should have the meaning given below. Other definitions are set forth throughout the application.

The term “about” can refer to a value or composition that is within an acceptable error range for a particular value or composition as determined by one of ordinary skill in the art, which will depend in part on how the value or composition is measured or determined. For example, as used herein, the expression “about 100” comprises all values between 99 and 101 (e.g., 99.1, 99.2, 99.3, 99.4, etc.).

As used herein, the terms “comprising” or “including” may be open, semi-closed, or closed. In other words, the terms also include “consisting essentially of” or “consisting of.”

Sequence identity is determined by comparing two aligned sequences along a predetermined comparison window (which can be 50%, 60%, 70%, 80%, 90%, 95% or 100% of the length of the reference nucleotide sequence or protein) and determining the number of positions at which identical residues occur. Typically, this is expressed as a percentage. The measurement of sequence identity of nucleotide sequences is a method well known to those skilled in the art.

As used herein, the terms “heavy chain variable region” and “V_{H}” can be used interchangeably.

As used herein, the terms “light chain variable region” and “V_{L}” can be used interchangeably.

As used herein, the terms “variable region” and “complementarity determining region (CDR)” are used interchangeably.

In the present invention, the terms “antibody of the present invention,” “protein of the present invention,” or “polypeptide of the present invention” are used interchangeably and refer to antibodies that specifically bind to CD27, such as proteins or polypeptides having a heavy chain variable region (e.g., the amino acid sequence of SEQ ID No: 27 or 35) and/or a light chain variable region (e.g., the amino acid sequence of SEQ ID No: 31 or 39). These may or may not contain an initial methionine.

### CD27

CD27, a co-stimulatory T cell receptor, co-stimulates with OX40 (CD134) and 4-1BB to promote the survival of activated T cells and is key to T cell priming and memory differentiation. Under normal circumstances, CD70 is primarily expressed on activated lymphocytes, but in pathological conditions, CD70 is highly expressed in a variety of tumor cells. Tumor cells express CD70 and bind to the T cell receptor CD27. Chronic co-stimulation causes T cells to express immune checkpoints such as PD-1 and TIM-3, leading to immune exhaustion. The high expression of CD70 in tumors may suggest that tumors use CD70 to control tumor-infiltrating lymphocytes (TILs) expressing CD27, thereby enabling immune escape. CD27-targeting antibodies have the mechanistic effect of enhancing tumor immunity and blocking immune escape.

### Antibody

As used herein, the term “antibody” refers to immunoglobulins, which are tetrapeptide chains composed of two identical heavy chains and two identical light chains connected by interchain disulfide bonds.

Existing antibody numbering schemes include:
1. The Kabat scheme (Kabat et al., 1991) is based on the location of regions of high sequence variation between sequences of the same domain type, with antibody heavy (VH) and light (Vλ and Vκ) variable domains numbered differently.
2. The Chothia program (Al-Lazikani, 1997) is identical to Kabat's, but corrects the position of the insertion annotation around the first VH complementarity-determining region (CDR) to correspond to the structural loop. Similarly, the enhanced Chothia program (Abhinandan and Martin, 2008) further structurally corrects the insertion position.
3. In contrast to these Kabat-like schemes, IMGT (Lefranc, 2003) and AHo (Honegger and Pluckthun, 2001) both define unique schemes for variable domains of antibodies and T-cell receptors (TCRs) (Vα and Vβ). Therefore, equivalent residue positions can be easily compared between domain types. IMGT and AHo differ in the number of positions they annotate (128 and 149, respectively) and the locations where they consider indels to occur.

The amino acid composition and arrangement order of the constant region of immunoglobulins (Ig) heavy chains vary, resulting in different antigenic properties. Consequently, immunoglobulins can be divided into five classes, or isotypes, namely IgM, IgD, IgG, IgA, and IgE, with their corresponding heavy chains being μ, δ, γ, α, and ε, respectively. Igs within the same class are further divided into subclasses based on the amino acid composition of their hinge regions and the number and location of disulfide bonds in the heavy chains. For example, IgG can be divided into IgG1, IgG2, IgG3, and IgG4. Light chains are classified as either κ or λ chains, depending on the constant region. Each of the five Ig classes can have either κ or λ chains. The subunit structures and three-dimensional configurations of different immunoglobulin classes are well known in the art.

The antibody light chain of the present invention may further comprise a light chain constant region, wherein the light chain constant region comprises a human or murine κ, λ chain or a variant thereof.

In the present invention, the antibody heavy chain described in the present invention may further comprise a heavy chain constant region, wherein the heavy chain constant region comprises human or murine IgG1, IgG2, IgG3, IgG4 or variants thereof. The sequence of approximately 110 amino acids near the N-terminus of the antibody heavy and light chains varies greatly and is the variable region (Fv region); the remaining amino acid sequence near the C-terminus is relatively stable and is the constant region. The variable region comprises three hypervariable regions (HVRs) and four framework regions (FRs) with relatively conserved sequences. The three hypervariable regions determine the specificity of the antibody and are also called complementarity determining regions (CDRs). Each light chain variable region (LCVR) and heavy chain variable region (HCVR) consists of three CDR regions and four FR regions, arranged in the order from amino terminus to carboxyl terminus: FRI, CDR1, FR2, CDR2, FR3, CDR3 and FR4. The three CDR regions of the light chain are LCDR1, LCDR2 and LCDR3; the three CDR regions of the heavy chain are HCDR1, HCDR2 and HCDR3. In the embodiment of the present invention, the six CDRs of the CD27 antibody are defined using the Kabat method.

The term “murine antibody” as used herein, refers to an anti-CD27 monoclonal antibody prepared with the knowledge and skill in the art. This is prepared by injecting a test subject with the CD27 antigen, followed by isolation of hybridomas expressing antibodies with the desired sequence or functional properties. In a preferred embodiment of the present invention, the murine CD27 antibody or antigen-binding fragment thereof may further comprise a light chain constant region of a murine κ or λ chain, or variants thereof, or a heavy chain constant region of a murine IgG1, IgG2, IgG3, or variants thereof.

The term “chimeric antibody” refers to an antibody formed by fusing the variable region of a mouse antibody with the constant region of a human antibody, which can reduce the immune response induced by the mouse antibody.

The term “humanized antibody”, also known as CDR-grafted antibody, refers to an antibody produced by transplanting mouse CDR sequences into the human antibody variable region framework, that is, different types of human germline antibody framework sequences. Humanized antibodies can overcome the heterologous reactions induced by chimeric antibodies due to the large amount of mouse protein components. Such framework sequences can be obtained from public DNA databases or published references including germline antibody gene sequences. In order to avoid a decrease in immunogenicity and the resulting decrease in activity, the human antibody variable region framework sequence can be subjected to minimal reverse mutation or back mutation to maintain activity.

The term “antigen-binding fragment of an antibody” (or simply “antibody fragment”) refers to one or more fragments of an antibody that retain the ability to specifically bind to an antigen (e.g., CLDN18.2). It has been shown that fragments of a full-length antibody can be used to perform the antigen-binding function of an antibody. Examples of binding fragments encompassed by the term “antigen-binding fragment of an antibody” include
(i) a Fab fragment, which is a monovalent fragment consisting of the VL, VH, CL, and CH1 domains;
(ii) a F(ab′)₂ fragment, which is a bivalent fragment comprising two Fab fragments linked by a disulfide bridge at the hinge region;
(iii) an Fd fragment consisting of the VH and CH1 domains;
(iv) an Fv fragment consisting of the VH and VL domains of a single arm of an antibody.

Fv antibodies contain the variable regions of the antibody heavy chain and light chain, but no constant region, and are the smallest antibody fragments with all antigen-binding sites. Generally, Fv antibodies also include a polypeptide linker between the VH and VL domains and are able to form the structure required for antigen binding.

The term “CDR” refers to one of the six hypervariable regions within the variable domain of an antibody that primarily contributes to antigen binding. One of the most commonly used definitions of the six CDRs is provided by Kabat E A et al. (1991) Sequences of proteins of immunological interest. NIH Publication 91-3242).

The term “epitope” or “antigenic determinant” refers to a site on an antigen to which an immunoglobulin or antibody specifically binds (e.g., a specific site on a CD27 molecule). An epitope typically includes at least 3, 4, 5, 6, 7, 8, 9, 10, 11, 12, 13, 14, or 15 consecutive or non-consecutive amino acids in a unique spatial conformation.

The terms “specific binding”, “selective binding”, “selectively binds” and “specifically binds” refer to the binding of an antibody to a predetermined epitope on an antigen. Usually, the antibody binds with an affinity (KD) of 10⁻⁷ M or less, for example, less than approximately 10⁻⁸ M, 10⁻⁹ M or 10⁻¹⁰.

The term “competitive binding” refers to an antibody that recognizes the same epitope (also referred to as an antigenic determinant) or a portion of the same epitope on the extracellular region of CD27 as the monoclonal antibody of the present invention and binds to the antigen. An antibody that binds to the same epitope as the monoclonal antibody of the present invention refers to an antibody that recognizes and binds to the amino acid sequence of CD27 recognized by the monoclonal antibody of the present invention.

The term “KD” or “Kd” refers to the dissociation equilibrium constant for a particular antibody-antigen interaction. Typically, the antibodies of the invention binds to CD27 with a dissociation equilibrium constant (KD) less than about 10⁻⁷ M, for example, less than about 10⁻⁸ M, 10⁻⁹ M or 10⁻¹⁰.

As used herein, the term “antigenic determinant” refers to a discrete three-dimensional site on an antigen that is recognized by the antibodies or antigen-binding fragments of the present invention.

The present invention includes not only complete antibodies, but also fragments of antibodies with immunological activity or fusion proteins formed by antibodies and other sequences. Therefore, the present invention also includes fragments, derivatives and analogs of the antibodies.

In the present invention, antibodies include murine, chimeric, humanized, or fully human antibodies prepared using techniques well known to those skilled in the art. Recombinant antibodies, such as chimeric and humanized monoclonal antibodies, including human and non-human portions, can be prepared using recombinant DNA techniques well known in the art.

As used herein, the term “monoclonal antibody” refers to an antibody secreted by a clone derived from a single cell. Monoclonal antibodies are highly specific, being directed against a single antigenic epitope. The cell may be a eukaryotic, prokaryotic, or phage clone.

In the present invention, the antibodies can be monospecific, bispecific, trispecific, or more multispecific.

In the present invention, the antibody of the present invention further comprises a conservative variant thereof, which means a polypeptide formed by replacing at most 10, preferably at most 8, more preferably at most 5, and most preferably at most 3 amino acids with amnio acid of similar properties as compared with the amino acid sequence of the antibody of the present invention. These conservative variant polypeptides are best produced by amino acid substitution according to the table below.

| | | Preferred |
|---|---|---|
| Initial residue | Representative substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

### Anti-CD27 Humanized Antibody

In another preferred embodiment, the heavy chain constant region and/or light chain constant region of the antibody of the present invention can be a humanized heavy chain constant region or light chain constant region. More preferably, the humanized heavy chain constant region or light chain constant region is a heavy chain constant region of human IgG1, IgG2, etc., or a human kappa or lambda light chain constant region.

In another preferred embodiment, the sequence formed by adding, deleting, modifying and/or replacing at least one amino acid sequence is preferably an amino acid sequence with a homology of at least 80%, preferably at least 85%, more preferably at least 90%, and most preferably at least 95%.

The antibody of the present invention may be a double-chain or single-chain antibody, and may preferably be a fully humanized antibody.

The antibody derivatives of the present invention can be single-chain antibodies and/or antibody fragments, such as Fab, Fab′, (Fab′)2, or other antibody derivatives known in the art, as well as any one or more of IgA, IgD, IgE, IgG, IgM antibodies or other subtypes of antibodies.

The antibody of the present invention may be a humanized antibody, a CDR-grafted and/or modified antibody targeting CD27.

In the above content of the present invention, the number of added, deleted, modified and/or substituted amino acids is preferably not more than 40% of the total number of amino acids in the initial amino acid sequence, more preferably not more than 35%, more preferably 1-33%, more preferably 5-30%, more preferably 10-25%, more preferably 15-20%.

### Preparation of Antibody

Any method suitable for producing monoclonal antibodies can be used to produce the CD27 antibodies of the present invention. For example, animals can be immunized with linked or naturally occurring CD27 proteins or fragments thereof. Suitable immunization methods can be used, including adjuvants, immunostimulants, repeated booster immunizations, and one or more routes can be used.

Any suitable form of CD27 can be used as an immunogen (antigen) to produce non-human antibodies specific for CD27 and screen the biological activity of the antibodies. The immunogen can be used alone or in combination with one or more immunogenicity enhancers known in the art. The immunogen can be purified from a natural source or produced in genetically modified cells. The DNA encoding the immunogen can be genomic or non-genomic (e.g., cDNA) in origin. The DNA encoding the immunogen can be expressed using a suitable genetic vector, including but not limited to adenoviral vectors, baculoviral vectors, plasmids, and non-viral vectors.

The sequence of the DNA molecule of the antibody of the present invention or its fragment can be obtained by conventional techniques, such as PCR amplification or genomic library screening. In addition, the coding sequences of the light chain and heavy chain can be fused together to form a single-chain antibody.

Once the relevant sequence is obtained, it can be obtained in large quantities by recombinant methods. This is usually done by cloning it into a vector, then transferring it into cells, and then isolating the relevant sequence from the propagated host cells by conventional methods.

In addition, artificial synthesis methods can also be used to synthesize relevant sequences, especially when the fragment length is relatively short. Generally, by first synthesizing multiple small fragments and then connecting them, very long fragments of sequence can be obtained. The DNA sequence can then be introduced into various existing DNA molecules (or vectors) and cells known in the art.

The term “nucleic acid molecule” refers to DNA molecules and RNA molecules. Nucleic acid molecules can be single-stranded or double-stranded, but are preferably double-stranded DNA. A nucleic acid is “operably linked” when it is placed into a functional relationship with another nucleic acid sequence. For example, a promoter or enhancer is operably linked to a coding sequence if it affects the transcription of the coding sequence.

The term “vector” refers to a nucleic acid molecule capable of transporting another nucleic acid to which it has been linked. In one embodiment, the vector is a “plasmid,” which refers to a circular double-stranded DNA loop into which additional DNA segments can be ligated.

The present invention also relates to vectors comprising the above-mentioned appropriate DNA sequence and appropriate promoter or control sequence. These vectors can be used to transform appropriate host cells to enable them to express proteins.

The term “host cell” refers to a cell into which an expression vector has been introduced. The host cell can be a prokaryotic cell, such as a bacterial cell, a lower eukaryotic cell, such as a yeast cell, or a higher eukaryotic cell, such as a plant or animal cell (e.g., a mammalian cell).

The steps of transforming host cells with recombinant DNA described in the present invention can be carried out using techniques well known in the art. The transformants obtained can be cultured using conventional methods, and the transformants express the polypeptide encoded by the gene of the present invention. Depending on the host cell used, conventional culture medium is used under appropriate conditions.

Typically, the transformed host cells are cultured under conditions suitable for expression of the antibodies of the present invention. The antibodies of the present invention are then purified using conventional immunoglobulin purification procedures, such as protein A-Sepharose, hydroxyapatite chromatography, gel electrophoresis, dialysis, ion exchange chromatography, hydrophobic chromatography, molecular sieve chromatography, or affinity chromatography, among other conventional separation and purification methods well known to those skilled in the art.

The resulting monoclonal antibodies can be characterized by conventional means. For example, the binding specificity of the monoclonal antibodies can be determined by immunoprecipitation or in vitro binding assays such as radioimmunoassay (RIA) or enzyme-linked immunosorbent assay (ELISA).

### Antibody Preparations

Antibodies exhibit varying stability in different formulation buffers, manifesting as changes in charge heterogeneity, degradation of antibody molecules, and aggregation. These changes in quality properties are related to the physical and chemical properties of the antibodies themselves. Therefore, during the antibody drug development process, it is necessary to screen formulation buffers suitable for each antibody based on its physical and chemical properties. Currently, commonly used antibody formulation buffer systems include phosphate buffer, citrate buffer, and histidine buffer. Depending on the properties of the antibody, varying concentrations of salt ions or excipients such as sorbitol, trehalose, and sucrose, as well as appropriate amounts of surfactants such as Tween, are added to maintain antibody stability.

### Pharmaceutical Composition

The present invention also provides a composition. In a preferred embodiment, the composition is a pharmaceutical composition, which contains the above-mentioned antibody or its active fragment or its fusion protein or its ADC or corresponding CAR-T cell, and a pharmaceutically acceptable carrier. Generally, these substances can be formulated in a non-toxic, inert and pharmaceutically acceptable aqueous carrier medium, wherein the pH is generally about 5-8, preferably about 6-8, although the pH value may vary with the properties of the formulated substance and the condition to be treated. The prepared pharmaceutical composition can be administered by conventional routes, including (but not limited to): intratumoral, intraperitoneal, intravenous, or local administration.

The antibody of the present invention can also be expressed in cells by nucleotide sequences for cell therapy, for example, the antibody is used in chimeric antigen receptor T cell immunotherapy (CAR-T) and the like.

The pharmaceutical composition of the present invention can be used to directly bind to CD27 protein molecules, and thus can be used to prevent and treat CD27-related diseases. In addition, other therapeutic agents can also be used simultaneously.

The pharmaceutical composition of the present invention contains a safe and effective amount (e.g., 0.001-99 wt %, preferably 0.01-90 wt %, more preferably 0.1-80 wt %) of the above-mentioned monoclonal antibody of the present invention (or its conjugate) and a pharmaceutically acceptable carrier or excipient. Such carriers include (but are not limited to): saline, buffer, glucose, water, glycerol, ethanol, and combinations thereof. The pharmaceutical preparation should match the mode of administration. The pharmaceutical composition of the present invention can be prepared in the form of an injection, for example, using physiological saline or an aqueous solution containing glucose and other adjuvants by conventional methods. Pharmaceutical compositions such as injections and solutions are preferably manufactured under sterile conditions. The dosage of the active ingredient is a therapeutically effective amount, for example, about 1 μg/kg body weight to about 5 mg/kg body weight per day. In addition, the polypeptide of the present invention can also be used in conjunction with other therapeutic agents.

When using a pharmaceutical composition, a safe and effective amount of the pharmaceutical composition is administered to a mammal, wherein the safe and effective amount is generally at least about 10 μg/kg body weight, and in most cases does not exceed about 50 mg/kg body weight. Preferably, the dosage is about 10 μg/kg body weight to about 20 mg/kg body weight. Of course, the specific dosage should also take into account factors such as the route of administration and the patient's health condition, which are all within the skill of a skilled physician.

### Detection Uses and Kits

The antibodies of the present invention can be used in detection applications, for example, for detecting a sample to provide diagnostic information.

In the present invention, the samples used include cells, tissue samples and biopsy specimens. The term “biopsy” as used in the present invention should include all types of biopsies known to those skilled in the art. Therefore, the biopsy used in the present invention can include, for example, tissue samples prepared by endoscopic methods or puncture or needle biopsy of an organ.

Samples used in the present invention include fixed or preserved cell or tissue samples.

The present invention also provides a kit containing the antibody (or fragment thereof) of the present invention. In a preferred embodiment of the present invention, the kit further comprises a container, instructions for use, a buffer, etc. The antibody of the present invention can also be fixed to a detection plate.

The main advantages of the present invention include:
(a) The present inventors obtained anti-CD27 mouse monoclonal antibodies 13B3, 16B1 and 17H3 with high specificity and high affinity through hybridoma fusion technology, and prepared human-mouse chimeric monoclonal antibodies and humanized antibodies based on these.
(b) The antibodies of the present invention have high affinity and specificity for human CD27 and can bind not only to solid-phase human CD27 but also to membrane-bound human CD27 with high efficiency.
(c) The antibodies of the present invention have significant in vitro activity. They not only efficiently bind to human CD27, efficiently activate T cells and promote T cell proliferation, but also efficiently block the binding of CD27 to CD70, thereby suppressing immunity and being used for the treatment of autoimmune diseases.
(d) The structure of the antibody of the present invention is stable.
(e) The humanized antibodies of the present invention have low or no immunogenicity when applied to humans.
(f) The antibodies of the present invention bind to the solid phase-bound cynomolgus monkey CD27 recombinant protein to some extent, but do not bind to mouse CD27, thus facilitating in vivo experiments in non-human primates.
(g) The antibodies of the present invention have ADCC activity and therefore have a significant specific killing effect on leukemias (myeloid leukemia, lymphocytic leukemia, T-cell leukemia, B-cell leukemia, etc.) and lymphomas that highly express CD27.
(h) The ACDD loss-of-function mutant antibody of the present invention has essentially no direct killing effect on T lymphocytes expressing CD27 and is suitable for applications requiring activation of T cell immune function and maintenance of T cell numbers, such as the treatment of solid tumors and non-T lymphocyte hematological tumors.

The present disclosure is further set forth below with reference to specific embodiments. The conditions of the experimental methods not specifically indicated in the following examples are usually in accordance with conventional conditions of Sambrook et al., Molecular Cloning: A Laboratory Manual (New York: Cold Spring Harbor Laboratory Press, 1989), or according to the conditions recommended by the manufacturer. Percentages and parts are by weight unless otherwise stated.

Unless otherwise specified, the materials and reagents used in the examples of the present invention are commercially available products.

### Example 1 Generation of Anti-Human CD27 Mouse Monoclonal Antibodies

### 1.1 Immunization

Five C57 mice were multi-point immunized intraperitoneally and subcutaneously with recombinant human CD27 protein in complete Freund's adjuvant (CFA). Booster immunizations were performed on days 14 and 21 with the same immunogen, CD27, in incomplete Freund's adjuvant.

### 1.2 Fusion

Mouse spleen cells were fused with mouse myeloma cells.

### 1.3 Hybridoma Screening

On days 5-10 after fusion, depending on the cell growth density, the medium was completely replaced with IMDM medium containing 10% FBS, 200 μl/well, incubated at 37° C., 5% CO₂. On days 10-14 after fusion, ELISA was performed depending on the cell growth density. Cells from the positive wells were subcloned for the first and second time. The testing steps are as follows:

Post-fusion binding antibody screening: CD27-mFc coating: CD27 was diluted to a concentration of 1 μg/ml in PBS, and mixed well, added into a 96-well ELISA plate at a concentration of 50 μl/well, sealed with sealing film and incubated at 4° C. overnight. ELISA plate blocking: The plate was washed twice with 250 μl/well of PBS, and patted dry. 150 μl/well of blocking solution was added, sealed with sealing film, and incubated at 37° C. for 1.5 hours. Primary antibody incubation: 100 μl/well of hybridoma supernatant was added to the ELISA plate, sealed with sealing film, and incubated at 37° C. for 1 hour. Secondary antibody incubation: the blocking solution was discarded, and the plate was washed three times with 250 μl of PBST, and patted dry. 100 μl/well of goat anti-mouse Fab HRP secondary antibody diluted 1:3000 in binding solution was added, sealed with sealing film, and incubated at 37° C. for 1 hour. TMB incubation: the blocking solution was discarded, and the plate was washed three times with 250 μl of PBST, and patted dry. 50 μL of TMB was added per well of the microplate, sealed with sealing film, and incubated at 37° C. for 15-25 minutes. The reaction was terminated with 50 μL of 2M HCl per well. Microplate Reader Detection: a microplate reader was detected at dual wavelengths of 450 nm and 655 nm, and the results were calculated by OD450-OD655.

### Competitive Antibody Assay:

CD27-mFc Coating: CD27-mFc was diluted in PBS to a concentration of 1 μg/ml. Mixed thoroughly and added into a 96-well ELISA using a multi-channel pipette at 50 μl/well. Sealed with sealing film and incubated at 4° C. overnight. Plate Blocking: the plated was washed twice with 250 μl/well PBS and patted dry. 150 μl/well of blocking solution was added, sealed with sealing film, and incubated at 37° C. for 1.5 hours. Primary Antibody Incubation: the plated was washed twice with 250 μl/well PBS and patted dry. CD70-hFc was diluted at 0.6 μg/ml in binding buffer and 50 μl/well was added to each 96-well dilution plate. 50 μl/well of hybridoma supernatant was added to the corresponding dilution plate, mixed thoroughly, and then 100 μl/well was added to the coated 96-well ELISA plate. Sealed with sealing film and incubated at 37° C. for 1.5 hours. Secondary antibody incubation: the blocking buffer was discarded, the plated was washed three times with 250 μl of PBST, and patted dry. AP anti-human Fc was diluted at a 1:3000 ratio in binding buffer, 100 μl/well was added to the ELISA plate, sealed with film, and incubated at 37° C. for 1 hour. PNPP incubation: the blocking buffer was discarded, washed three times with 250 μl of PBST, and patted dry. One PNPP pellet was dissolved in 1 ml of 5× diethanolamine substrate, then 4 ml of deionized water was added. 50 μl/well was added to the ELISA plate, sealed with film, and incubated at 37° C. for 15-25 minutes. Microplate Reader Detection: Dual wavelength detection at 405 nm/490 nm, with results calculated as OD405-OD490.

### FACS Assay:

An expression cassette for expressing human CD27 was introduced into CHO cells to obtain a CHO cell line expressing human CD27 (transmembrane protein), which was designated as CHO-CD27.

CHO-CD27 cells was counted and centrifuged at 1000 rpm for 5 minutes, resuspended in 0.5% BSA PBS and 5E5 cells/well was added to a 96-well conical-bottom plate. The plated was centrifuged at 400 g for 5 minutes and the supernatant was discarded. Two subclones were selected from each positive clone and 100 μl/well of supernatant was added to a 96-well conical-bottom plate, incubated at 4° C. for 30 minutes. The plate was washed twice with 250 μl/well of 0.5% BSA PBS. 100 μl/well of anti-mouse IgG-FITC was added to a 96-well conical-bottom plate, incubated at 4° C. for 30 minutes. The plated was washed twice with 250 μl/well of 0.5% BSA PBS and 200 μl/well of PBS was added to the plate. Samples were analyzed using a Beckman CytoFLEX.

### Experimental Results and Analysis:

From the numerous clones, three clones with excellent performance were selected. These three monoclonal antibodies all bind to solid-phase human CD27 and membrane-bound human CD27, and have some binding to solid-phase cynomolgus macaque CD27 recombinant protein, but do not bind to mouse CD27. The three clones are 13B3, 16B1, and 17H8. Among them, 13B3 and 16B1 have the function of significantly blocking CD70 binding to CD27, while 17H8 has a weaker blocking ability.

### 1.4 Heavy and Light Chain Variable Region Sequencing

1.4.1 RNA Extraction and cDNA Acquisition

About 1×10⁵ to 2×10⁵ hybridoma cells was taken to extract RNA (Sigma, GenElute™ Mammalian Total RNA Miniprep Kit, Catalog No. RTN70). Approximately 8 μL of total RNA was used for subsequent reverse transcription PCR. First, the RNA was incubated at 65° C. for 5 minutes to eliminate RNA secondary structures. Then, 2 μL of 5×gDNA wiper was added at 42° C. for 2 minutes to remove genomic DNA contamination. Finally, 2 μL of 10× RT Mix, 2 μL of HiscriptIII Enzyme Mix, and 1 μL of random hexamers (Random Hexamers, Norvegren, R312) were added. The tube was gently tapped to mix thoroughly, centrifuged briefly to remove the liquid from the tube bottom. Finally, the reverse transcription reaction was performed under the following conditions: 25° C. for 5 minutes; 42° C. for 45 minutes; and 85° C. for 5 seconds.

### 1.4.2 Amplification of Antibody Variable Region Genes

The above cDNA was used as a template and PCR was used to amplify the hybridoma antibodies with different primer combinations. The primers used in this experiment mainly refer to the literature von Boehmer, L. et al. Sequencing and cloning of antigen-specific antibodies from mouse memory B cells. Nat Protoc, 2016.11(10): p.1908-1923.

Reaction conditions: denaturation at 94° C. for 3 minutes, 40 cycles of 94° C. for 30 seconds, 55° C. for 30 seconds, and 72° C. for 35 seconds, with incubation at 72° C. for 5 minutes at the end of the cycle.

In the early stage of the present invention, three mouse hybridomas that bind to solid phase and membrane forms of CD27 were screened, namely 13B3, 16B1 and 17H8. Each clone was further cloned and the heavy chain and light chain V region sequence analysis was performed.

Results: The heavy and light chain PCR bands obtained were bright and of the correct size, and the sequencing reaction peaks were normal and single. NCBI Blast results showed that they were consistent with normal antibody V region sequence characteristics, which have complete Framework and CDR regions, without stop codons, without frameshift mutations, and have VDJ/VJ gene rearrangements. The V regions of all antibodies were successfully obtained, with the 13B3 and 16B1 sequences being identical. The specific sequences are shown in Table 1 below:

TABLE 1

| | | | SEQ | |
|---|---|---|---|---|
| Clone | Description | | ID | Sequence |
| 13B3 | VH | Full-length | 1 | QVQLQQSGPEQVKPGASVKISCKTSGYAFSQSWM |
| (16B1-1-1) | | sequence | | NWVKQRPGQGLEWIGRIYPGDGDTNYNGIFKGKA |
| (Blocking | | | | TLTADKSSSTAYMQLSSLTSLDSAVYFCASMIRT |
| type) | | | | TYVMDHWGQGTSVTVTS |
| | | CDR1 | 2 | QSWMN |
| | | CDR2 | 3 | RIYPGDGDTNYNGIFKG |
| | | CDR3 | 4 | MIRTTYVMDH |
| | VL | Full-length | 5 | DIVLTQSPATLSVTPGDSVSLSCRASQSVSNSLHW |
| | | sequence | | YQQKSHESPRLLIMYTSQSISGIPSRFSGSGSGTD |
| | | | | FTLSINSVETEDFGMYFCQQSNSWPLTFGAGTKLE |
| | | | | LK |
| | | CDR1 | 6 | RASQSVSNSLH |
| | | CDR2 | 7 | YTSQSIS |
| | | CDR3 | 8 | QQSNSWPLT |
| 17H8 | VH | Full-length | 9 | EVKLEESGGGLVQPGGSMKLSCVASGFTFSNYW |
| (17H8-1-1) | | sequenc | | MNWVRQSPEKGLEWVAEIRLKSNNYATYYAESV |
| | | | | KGRFTISRDDSKSSVYLQMNSLRGEDTGIYYCTLY |
| | | | | DSSYYYAMDYWGQGTSVTVSS |
| | | CDR1 | 10 | NYWMN |
| | | CDR2 | 11 | EIRLKSNNYATYYAESVKG |
| | | CDR3 | 12 | YDSSYYYAMDY |
| | VL | Full-length | 13 | DVVLTQTPLSLPVNIGDQASISCKSTQSLLHSDGFT |
| | | sequenc | | YLDWYLQKPGQSPQLLIYLVSNRFLGVPDRFSGSG |
| | | | | SRTDFTLKISRVEAEDLGVYYCFQSNYLPLTFGGG |
| | | | | TKLEI |
| | | CDR1 | 14 | KSTQSLLHSDGFTYLD |
| | | CDR2 | 15 | LVSNRFL |
| | | CDR3 | 16 | FQSNYLPLT |

### Example 2. Preparation of Human-Mouse Chimeric Monoclonal Antibodies

Previous sequencing analysis revealed the sequences of three anti-human CD27 murine hybridoma antibodies, two of which shared identical sequences. In this experiment, two clones with different sequences were recombinantly expressed and their activity was analyzed. Specific PCR primers were designed based on the different sequences. The PCR reaction conditions were as follows: denaturation at 94° C. for 3 minutes, followed by 40 cycles of 94° C. for 30 seconds, 55° C. for 30 seconds, and 72° C. for 35 seconds, followed by incubation at 72° C. for 5 minutes at the end of each cycle.

The heavy chain expression vector (plasmid P378) and the light chain expression vector (P379) were digested with Xho I. The digested vectors were identified by agarose gel electrophoresis. After confirmation of the correct bands, they were recovered and purified. The digested vector and fragments were quantified and homologously ligated. The ligated products were transformed, and the plasmids were extracted for transient cell transfection. All 293F cells, culture medium, and transfection reagents for this experiment were purchased from Sino Biological. The culture medium of the transiently transfected cells was centrifuged at 4000 rpm for 10 minutes, and the cell pellet was discarded. An appropriate amount of protein A beads was added to the supernatant and incubated at room temperature for 4 hours. After centrifugation and discarding the supernatant, the protein A beads were washed twice with PBS and eluted with citric acid (pH=3). Finally, the pH was adjusted to neutral with 1M Tris. The purified protein was analyzed by SDS-PAGE.

The results are shown in FIG. 1, and the reduced and non-reduced band sizes of each antibody were normal.

### Example 3. Binding of Human-Mouse Chimeric CD27 Monoclonal Antibody to Antigen CD27

### 3.1 Study on the In Vitro Binding Activity of Human-Mouse Chimeric CD27 Monoclonal Antibody to Antigen CD27 Using ELISA

In order to determine whether the human-mouse chimeric monoclonal antibody of the present disclosure can bind to the CD27 antigen, an in vitro test was performed, and the CD27 monoclonal antibody Varlilumab (1F5) was used as a positive control.

Binding of two CD27 monoclonal antibodies to the CD27 antigen (Kangdai) was tested by ELISA using standard methods and procedures. The specific steps were as follows: the antigen CD27-mFc (Kangdai) was diluted to 1 μg/mL in NaHCO₃ solution (pH 9.6), and 50 μL was added to each well of a 96-well microtiter plate and refrigerated at 4° C. overnight. The next day, after washing twice with PBS, the plate was blocked with 3% BSA and 100 μL was added to each well for 1.5 hours at 37° C. After washing four times with PBST, the plate was added with antibody diluent and incubated at 37° C. for an additional 2 hours. After washing four more times with PBST, a secondary antibody (Ap-anti-human IgG Fc, Jackson Immuno Research, 109-055-098) was added at a predetermined dilution and incubated at 37° C. for 1.5 hours. Finally, after washing four times with PBST, the plate was added with PNPP colorimetric solution and incubated at 37° C. for 10-15 minutes before reading on a microtiter plate. 1F5 was used as a control antibody.

### Experimental Results and Analysis:

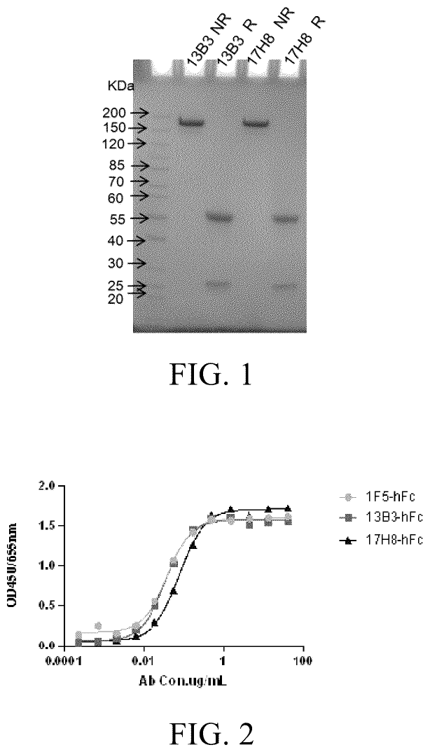

ELISA test results showed (FIG. 2, Table 2): Two CD27 human-mouse chimeric monoclonal antibodies (numbered 13B3-hFc and 17B8-hFc) can specifically bind to CD27. The binding affinity EC₅₀ of the positive control 1F5 was 0.0381 μg/mL. The results showed that the recombinantly expressed 7104 chimeric antibody had a good binding activity to CD27, which was comparable to that of the positive control 1F5.

TABLE 2

| | EC₅₀(ug/mL) |
|---|---|
| 1F5-hFc (positive control) | 0.0381 |
| 13B3-hFc | 0.0344 |
| 17H8-hFc | 0.0754 |

### 3.2 Study of the Binding of Human-Mouse Chimeric CD27 Monoclonal Antibody to CD27 Protein on Cell Membrane Using Flow Cytometry

In order to determine whether the human-mouse chimeric monoclonal antibody of the present disclosure can bind to the CD27 protein on the CHO cell membrane, an in vitro test was performed, using 1F5 as a positive control.

First, FACS buffer was prepared, i.e., 1×PBS+0.5% BSA solution. CHO cell line that stably and highly expresses human CD27 was taken, with the amount of cells required for one sample approximately 1-5×10⁵, The antibody at a certain ratio was diluted with FACS buffer and the cells were resuspended in a volume of about 100 μL, incubated in a 4° C. refrigerator for 30 min, the cells were washed once with FACS buffer, the secondary antibody FITC-anti-human IgG (Abcam: 6854) was diluted with FACS buffer, the cells were resuspended and incubated in a 4° C. refrigerator for 30 min, and finally, the cells were washed with FACS buffer, resuspended with 200 μL FACS buffer and detected on the machine.

TABLE 3

| | EC₅₀(ug/mL) |
|---|---|
| 1F5-hFc (positive control) | 2.869 |
| 13B3-hFc | 2.486 |
| 17H8-hFc | ~90.61 |

### Experimental Results and Analysis:

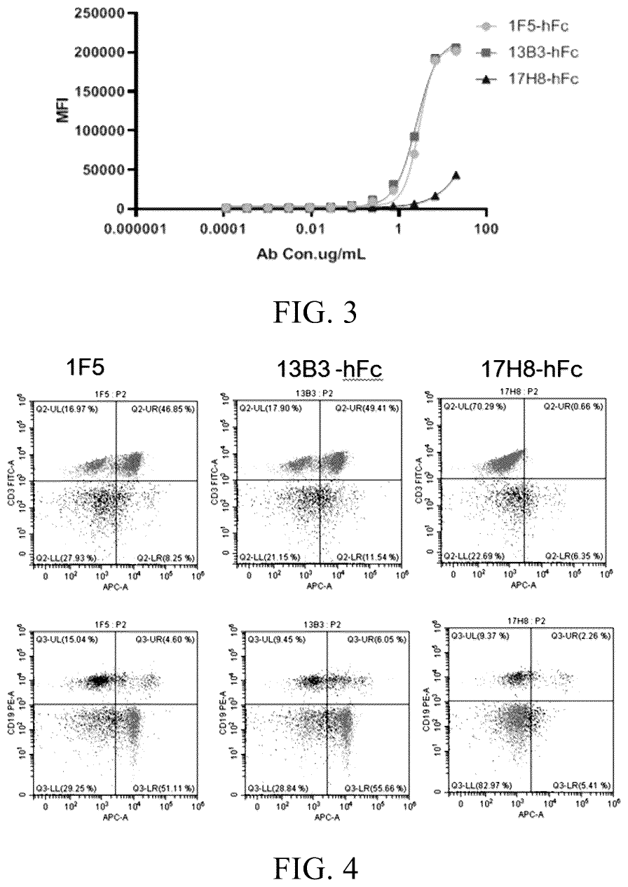

The results are shown in FIG. 3 and Table 3. The 13B3-hFC chimeric monoclonal antibody exhibits favorable binding to CHO-CD27, with an EC₅₀ of 2.486 μg/mL for CHO-CD27 binding. The positive control 1F5 shows an EC₅₀ of 2.869 μg/mL, indicating comparable binding affinity. This data demonstrates that the 13B3-hFc antibody of the present invention not only has strong binding capacity to solid-phase antigens (ELISA method), but also has high binding activity to CD27 on the cell membrane. In contrast, the 17H8-hFc chimeric monoclonal antibody binds poorly to CHO-CD27.

### 3.3 FACS Detection of the Binding of Human-Mouse Chimeric CD27 Antibody to CD27 Protein on the Surface of Human T and B Cells

In order to determine whether the human-mouse chimeric monoclonal antibody 7104 of the present disclosure can bind to the CD27 protein on the membrane of human T and B cells, an in vitro test was performed, using 1F5 as a positive control.

Peripheral blood mononuclear cells were isolated from freshly collected human blood by density gradient centrifugation. Approximately 5×10\hat{ }5 cells per sample in 100 μL (PBS+0.5% BSA) were added with the test antibodies 1F5, 13B3-hFc, and 17H8-hFc, respectively, at a final concentration of 5 μg/mL. The cells were incubated at 4° C. for 30 min. The cells were washed once with FACS working buffer (PBS+0.5% BSA). The cells were resuspended in 100 μL of FACS working buffer and the secondary antibodies Cy5-AffiniPure F(ab′)₂ Fragment Goat Anti-Human IgG, Fcγ fragment specific (Jackson Immuno Research, 109-176-098), FITC-anti-human CD3 antibody (BD Bioscience, 556611), and PE-anti-human CD19 (biolegend) were added. After gentle mixed, the cells were incubated at 4° C. for 30 min. Finally, the cells were washed with FACS working solution, resuspended in 300 μL buffer, and detected on the machine.

### Experimental Results:

The results are shown in FIG. 4. The first row shows the expression of CD27 on CD3+ T cells, and the second row shows the expression of CD27 on CD19+ B cells.

The results showed that CD27 expression was high on T cells, approximately 75%, while expression was lower on B cells, approximately 20%, 13B3 was able to bind well to both T and B cells, with staining comparable to the control 1F5, while 17H8 was less able to bind to both T and B cells.

### 3.4 Using Biolayer Interferometry (BLI) to Study the Binding Activity of Human-Mouse Chimeric CD27 Monoclonal Antibodies to CD27

The dissociation constant (Kd) of the chimeric CD27 monoclonal antibody was determined using a GATOR (ProbeLife) instrument and an AHC (Pall, 185064) Human Antibody Capture probe. The chimeric CD27 monoclonal antibody was diluted to 50 nM in binding buffer (Q buffer [PBS (10 mM pH 7.4)+0.02% Tween 20+0.2% BSA]. The chimeric CD27 antibody was serially diluted two-fold in Q buffer, starting with a maximum concentration of 100 nM. Kinetic association assays were initiated by placing the antibody capture sensor in the serially diluted antigen solution. Octet Data Acquisition software was opened and the New Kinetics Experiment-Basic Kinetics mode was selected;

The process was set up following the table below:

TABLE 4
Procedure for studying CD27 antibody binding
activity using biofilm interferometry (BLI)

| Process | Time | | Concentration |
|---|---|---|---|
| Baseline 1 | 30 | seconds | Q buffer |
| Loading | 120 | seconds | 30 nM antibody (in Q buffer) |
| Baseline 2 | 60 | seconds | Q buffer |
| Association | 120 | seconds | Antibody in Q Buffer, starting from |
| | | | 100 nM highest concentration with |
| | | | two-fold serial dilution |
| Dissociation | 120 | seconds | Q buffer |
| Regeneration | 25 | seconds | Q buffer |

TABLE 5
Kinetic analysis results of human-mouse chimeric CD27
monoclonal antibodies binding to antigen CD27

| Sample ID | KD (M) | kon(1/Ms) | kdis(1/s) | Full R\hat{ }2 | RMax |
|---|---|---|---|---|---|
| 13B3 | 1.227E−09 | 1.81E+05 | 2.22E−04 | 0.9992 | 0.4909 |
| 17H8 | 1.301E−09 | 1.23E+05 | 1.60E−04 | 0.9977 | 0.1989 |
| 1F5 (positive | 6.962E−10 | 1.26E+05 | 8.75E−05 | 0.998 | 0.2531 |
| control) | | | | | |

As shown in the table above, the correlation coefficient R2 for all antibodies in the global fitting mode was greater than 0.95, meeting the system suitability criteria and confirming the reliability of the results.

### 3.5 Inhibition of the Binding of CD27 and CD70 by Human-Mouse Chimeric CD27 Antibodies

In order to determine whether the human-mouse chimeric CD27 monoclonal antibody of the present disclosure can inhibit the binding of CD27 to its ligand CD70, an in vitro ELISA test was performed.

CD70 (R&D, 9328-CL-100) was diluted to 1 μg/mL in PBS, and 50 μL was added to each well of a 96-well microtiter plate and refrigerated at 4° C. overnight. The next day, the plates were washed twice with PBS and blocked with 3% BSA (100 μL per well) for 1.5 hours at 37° C. After four washes with PBST, a fixed concentration of CD27-mFc (final concentration of 4.3 μg/mL) and antibody diluent were added, followed by an additional 2 hours of incubation at 37° C. After four more washes with PBST, a 1:300 dilution of secondary antibody, HRP-anti-mouse Fc (Jackson Immuno Research, 115-035-164), was added and incubated at 37° C. for 1 hour. Finally, after four washes with PBST, TMB colorimetric solution (Biyuntian, P0209) was added and incubated at 37° C. for 10-15 minutes. The plates were then terminated with 2M HCl and read on a microtiter plate.

TABLE 6

| | IC₅₀ (ug/mL) |
|---|---|
| 13B3-hFc | 1.474 |
| 17H8-hFc | NA |

### Experimental Results:

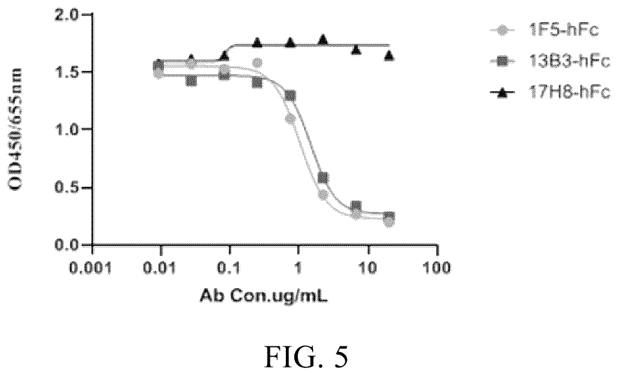

The experimental results are shown in Table 6 and FIG. 5, 13B3-hFc chimeric CD27 monoclonal antibody can specifically block the binding of CD27 and CD70, with an IC₅₀ of 1.474 ug/mL.

17H8-hFc did not block the binding of CD27 to CD70, suggesting that the binding site of 17H8-hFc to CD27 is different from that of 13B3-hFc.

### Example 4. Analysis of Binding Epitopes of Human-Mouse Chimeric CD27 Monoclonal Antibodies

### 4.1 Kinetic Binding Method Study

The method is the same as the affinity determination in Example 3.4, except that after the antigen CD27 was loaded, different antibodies were loaded. If the antibodies can continue to bind, it means that the epitopes of the two antibodies do not overlap. If they cannot continue to bind, it means that the epitopes overlap or are the same epitope.

### Experimental Results and Analysis:

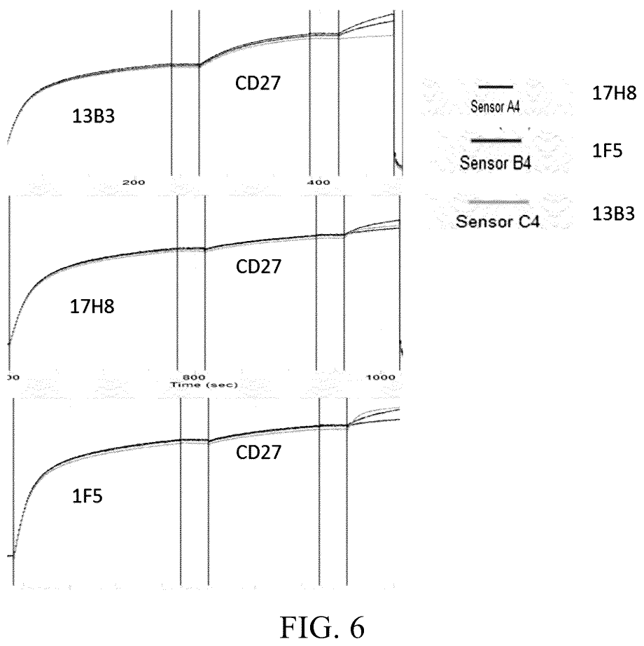

The results as shown in FIG. 6, showing that after 13B3 bound to the probe, the antigen CD27 was subsequently loaded, and the antigen was bound. However, when different antibodies were subsequently loaded, 13B3 could no longer bind, while 1F5 and 17H8 could continue to bind, indicating that 13B3 and 17H8/1F5 bind to different epitopes on CD27. Similarly, the same experimental results were obtained when 17H8 or 1F5 was bound to the probe, indicating that 13B3, 17H8, and 1F5 bind to different epitopes on CD27.

### 4.2 ELISA Method Study

13B3-hFc was diluted to 2 μg/mL in NaHCO₃ solution (pH 9.6). 50 μL was added to each well of a 96-well microtiter plate and refrigerated at 4° C. overnight. The next day, the plates were washed twice with PBS and blocked with 3% BSA (100 μL per well) for 1.5 hours at 37° C. After four washes with PBST, a fixed concentration of CD27-mFc (final concentration 0.8 μg/mL) and 1F5 antibody dilution were added, followed by an additional 2 hours at 37° C. After four more washes with PBST, a 1:3000 dilution of HRP-anti-mouse Fc secondary antibody (Jackson Immuno Research, 115-035-164) was added and incubated at 37° C. for 1 hour. Finally, after four washes with PBST, TMB colorimetric solution (Biyuntian, P0209) was added and incubated at 37° C. for 10-15 minutes. The plates were then terminated with 2M HCl and read on a microtiter plate.

### Experimental Results and Analysis:

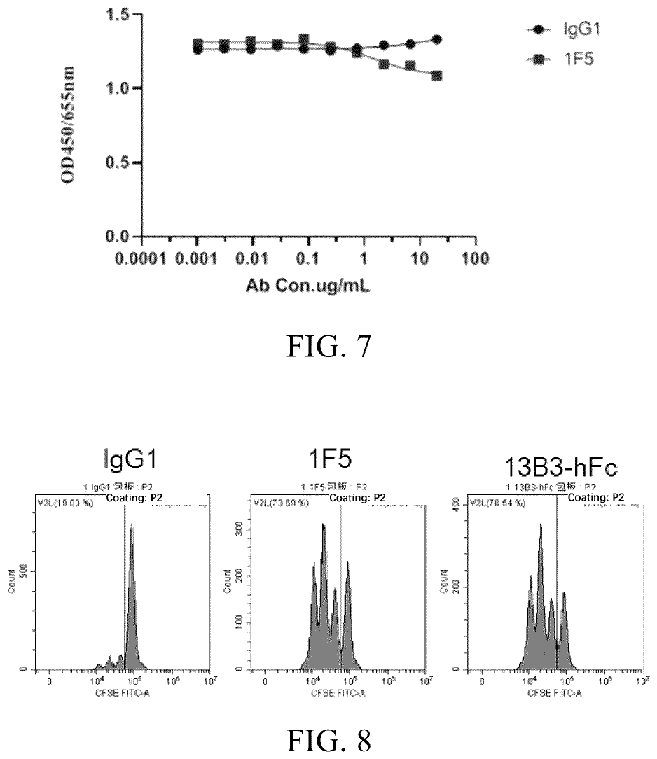

As shown in FIG. 7, compared with IgG1, the binding between CD27 and 13B3 was not significantly weakened even at a higher concentration of 1F5, indicating that the epitopes bound by 13B3 and 1F5 are different.

### Example 5. Human-Mouse Chimeric CD27 Monoclonal Antibody Promotes IFNγ Secretion and T Cell Proliferation In Vitro

### 5.1 Promotion of IFNγ Secretion

After T cell activation, lymphocytes secrete the cytokine IFNγ, which also promotes cell proliferation. To test the function of the disclosed human-mouse chimeric CD27 monoclonal antibody as a positive regulator of T cell activation, we conducted the following experiments, demonstrating the disclosed antibody has enhanced effect on IFNγ production in PBMCs and effect on T cell proliferation.

The day before the experiment, the test antibodies were diluted to 30 μg/mL in PBS. 50 μL per well was added to the corresponding wells of a 96-well plate and incubated at 4° C. overnight. The next day, the 96-well plate was taken, the PBS was discarded from the wells, and placed in a clean bench. PHA (Sigma, Cat. No. L9019) was diluted to 2 μg/mL in complete culture medium (RPMI1640+10% FBS) and set aside. PBMCs (Oricells) were resuscitated by taking the cells from the liquid nitrogen tank, quickly thawed in a 37° C. water bath, transferred to a 15 mL centrifuge tube, and centrifuged at 1000 rpm for 5 minutes. The supernatant was discarded and the cell density was adjusted to 5×10\hat{ }6/mL. 100 μL of the PHA dilution was transferred to the 96-well plate, followed by 100 μL of the cell suspension to the corresponding wells. After gentle shaken, incubated in a 37° C., 5% CO₂ incubator for 72 hours. After 72 h, the cell supernatant was collected and the IFNγ content was detected using an IFNγ detection kit (Biolegend, 430115).

### Experimental Conclusions and Analysis:

The results of supernatant IFNγ showed that compared with the blank control, 13B3 antibody could significantly promote the production of cellular IFNγ, with a change fold of 52 times. Compared with the positive control 1F5, the change fold of IFNγ release of 17H8 was 38, slightly lower than the positive control 1F5.

TABLE 7
IFNγ detection results

| | IFNγ (pg/mL) | Fold Change (FC) |
|---|---|---|
| Blank Control | 1250 | 1 |
| 1F5 (positive control) | 50895 | 41 |
| 13B3 | 64789 | 52 |
| 17H8 | 47000 | 38 |

### 5.2 Promote T Cell Proliferation

On the day prior to the experiment, anti-CD3 antibody (Biolegend, 317303) and the test antibodies were diluted to fixed concentrations using PBS. Then, 50 μL of the solution was added to each well of a cell culture plate. After gentle mixing, the plate was placed at 4° C. overnight. The following day, the PBS-antibody dilution was aspirated and discarded. The plate was left uncovered in a biosafety cabinet for several minutes to allow residual liquid to evaporate. Purified CD3⁺ T cells (Hycells, donor ID: PBZ1017) were thawed and centrifuged to collect the cell pellet. The cells were then labeled with CFSE (Beyotime, C0051) according to the manufacturer's instructions. Subsequently, the cells were resuspended in complete medium and adjusted to a density of 0.5×10⁶/mL. Then, 200 μL of the cell suspension was added to each well of the pre-coated 96-well plate. The plate was incubated at 37° C. in a 5% CO₂ incubator for 72 hours.

After 72 h, cells were collected to detect cell proliferation.

### Experimental Conclusions and Analysis:

As shown in FIG. 8, anti-CD27 antibodies can significantly promote the proliferation of CD3⁺ T cells. Compared with the control IgG1 (cell proliferation ratio is 19.03%), the proliferation ratios of cells in the 1F5 and 13B3-hFc treatment groups were 73.69% and 78.54%, respectively. Among them, the effect of 13B3-hFc was better than that of 1F5.

### Example 6. Blockade of CD70-CD27 Downstream Signaling Pathway by Human-Mouse Chimeric CD27 Monoclonal Antibody

Two cell lines were used in this experiment: 1. Jurkat-CD27-NFκB-Luc cells. The specific cell construction method is as follows: Jurkat cells were simultaneously electroporated with two plasmids: one expressing CD27 (CD27 cDNA constructed into a conventional expression plasmid driven by the CMV promoter) and the other containing the pNL3.2.NF-κB-RE [NlucP/NF-κB-RE/Hygro] Vector (Promega, N111A). After two weeks of selection with hygromycin and G418 pressure, CD27-positive single cells were sorted by flow cytometry. After the single cells grew to a population, they were plated onto cell culture plates coated with an anti-CD3 mAb gradient. After 16 hours, the cells were assayed (Promega, N1120) to select cell lines with dose-dependent expression and to simultaneously measure CD27 expression. Cell lines with high CD27 expression were selected for downstream experiments. 2. Raji cells: This cell line is a lymphoma cell line that highly expresses molecules such as CD80, CD86, and CD70.

The experimental steps are as follows: anti-CD27 mAbs were prepared into a 2× working solution, diluted to 60 ug/mL in complete culture medium (RPMI1640+10% FBS), then performed a 3-fold serial dilution, and 50 μL was added to a dedicated 96-well plate (Cornning, 3903). Then, 50 μL of a mixture of Jurkat-CD27-NFκB-Luc and Raji cells (number ratio of 2:1, density of 4E6/mL) was added to the culture plate and detected after 16 hours (Promega, N1120).

### Experimental Results and Analysis:

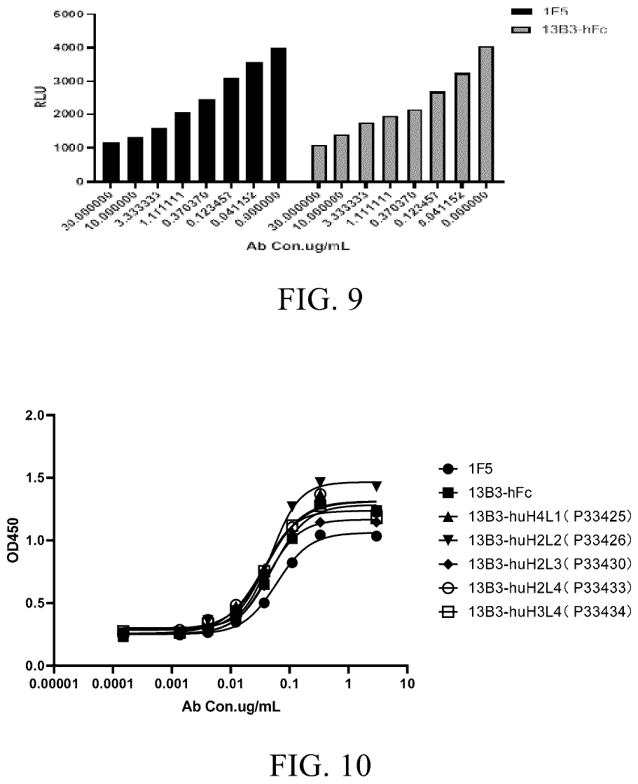

As shown in FIG. 9, anti-CD27 antibodies inhibit the CD27 downstream NFκB signaling pathway by blocking the binding of CD70 on Raji cells and CD27 on Jurkat cells. This effect becomes more pronounced with increasing antibody concentration. Although 1F5 and 13B3-hFc bind to different epitopes on CD27, they both exhibit this function with comparable efficacy.

### Example 8. Design and Preparation of Humanized CD27 Monoclonal Antibody

### 8.1 Design of Humanized CD27 Monoclonal Antibody

Based on the 13B3 mouse parent antibody sequence, a humanized antibody sequence was designed. The specific steps are as follows: First, use Discovery Studio and Schrödinger Antibody Modeling uses homology modeling to construct a three-dimensional molecular model of the variable region. Next, by comparing existing antibody structures in the database, structural simulations are performed on the variable region and CDR of the parent antibody. At the same time, cDNA-derived human germline sequences with high homology to the murine parent antibody VH and VL are selected for comparison. The 13B3 heavy chain VH uses IGHV1 with the highest homology as the humanization design template, and the sequence is designed. The light chain VL uses IGKV6 and IGKV3 as the humanization design template, and the sequence is designed.

### Result

The original heavy chain mVH sequence of the murine parent antibody numbered 13B3 was designed into four humanized sequences: 13B3-huVH1 (SEQ ID No: 17), 13B3-huVH2 (SEQ ID No: 18), 13B3-huVH3 (SEQ ID No: 19), and 13B3-huVH4 (SEQ ID No: 20). The original light chain mVL sequence of the antibody was designed into four humanized sequences: 13B3-huVL1 (SEQ ID No: 21), 13B3-huVL2 (SEQ ID No: 22), 13B3-huVL3 (SEQ ID No: 23), and 13B3-huVL4 (SEQ ID No: 24).

TABLE 8
VH and VL sequences of humanized antibodies based on 13B2

| | | SEQ ID | |
|---|---|---|---|
| Description | | No | Sequence |
| 13B3- | Full- | 17 | QVQLQQSGAEVKKPGASVKISCKTSGYAFSQSWMNWV |
| huVH1 | length | | KQAPGQGLEWIGRIYPGDGDTNYNGIFKGRATLTADKSS |
| | | | STAYMELSRLRSDDTAVYYCASMIRTTYVMDHWGQGTT |
| | | | VTVSS |
| | CDR | 2 | QSWMN |
| | 1 | | |
| | CDR | 3 | RIYPGDGDTNYNGIFKG |
| | 2 | | |
| | CDR | 4 | MIRTTYVMDH |
| | 3 | | |
| 13B3- | Full- | 18 | QVQLVQSGAEVKKPGASVKVSCKTSGYAFSQSWMNWV |
| huVH2 | length | | RQAPGQGLEWIGRIYPGDGDTNYNGIFKGRVTLTADKSS |
| | | | STAYMELSRLRSDDTAVYYCASMIRTTYVMDHWGQGTT |
| | | | VTVSS |
| | CDR | 2 | QSWMN |
| | 1 | | |
| | CDR | 3 | RIYPGDGDTNYNGIFKG |
| | 2 | | |
| | CDR | 4 | MIRTTYVMDH |
| | 3 | | |
| 13B3- | Full- | 19 | QVQLVQSGAEVKKPGASVKVSCKTSGYAFSQSWMNWV |
| huVH3 | 1ength | | RQAPGQGLEWIGRIYPGDGDTNYNGIFQGRVTLTADTSIS |
| | | | TAYMELSRLRSDDTAVYYCASMIRTTYVMDHWGQGTT |
| | | | VTVSS |
| | CDR | 2 | QSWMN |
| | 1 | | |
| | CDR | 25 | RIYPGDGDTNYNGIFQG |
| | 2 | | |
| | CDR | 4 | MIRTTYVMDH |
| | 3 | | |
| 13B3- | Full- | 20 | QVQLVQSGAEVKKPGASVKVSCKTSGYAFSQSWMNWV |
| huVH4 | length | | RQAPGQGLEWMGRIYPGDGDTNYAQKFQGRVTMTADT |
| | | | SISTAYMELSRLRSDDTAVYYCASMIRTTYVMDHWGQG |
| | | | TTVTVSS |
| | CDR | 2 | QSWMN |
| | 1 | | |
| | CDR | 26 | RIYPGDGDTNYAQKFQG |
| | 2 | | |
| | CDR | 4 | MIRTTYVMDH |
| | 3 | | |
| 13B3- | Full- | 21 | EIVLTQSPDFLSVTPKEKVTLTCRASQSVSNSLHWYQQKP |
| huVL1 | length | | DQSPKLLIMYTSQSISGIPSRFSGSGSGTDFTLTINSVEAED |
| | | | AATYYCQQSNSWPLTFGGGTKLELK |
| | CDR | 6 | RASQSVSNSLH |
| | 1 | | |
| | CDR | 7 | YTSQSIS |
| | 2 | | |
| | CDR | 8 | QQSNSWPLT |
| | 3 | | |
| 13B3- | Full- | 22 | IVLTQSPDFQSVTPKEKVTITCRASQSVSNSLHWYQQKPD |
| huVL2 | length | | QSPKLLIMYTSQSISGVPSRFSGSGSGTDFTLTINSLEAED |
| | | | AATYYCQQSNSWPLTFGGGTKLELK |
| | CDR | 6 | RASQSVSNSLH |
| | 1 | | |
| | CDR | 7 | YTSQSIS |
| | 2 | | |
| | CDR | 8 | QQSNSWPLT |
| | 3 | | |
| 13B3- | Full- | 23 | DIVLTQSPATLSVSPGERVTLSCRASQSVSNSLHWYQQKP |
| huVL3 | length | | GQAPRLLIMYTSQSISGIPARFSGSGSGTDFTLTISSVEPED |
| | | | FAVYYCQQSNSWPLTFGGGTKLELK |
| | CDR | 6 | RASQSVSNSLH |
| | 1 | | |
| | CDR | 7 | YTSQSIS |
| | 2 | | |
| | CDR | 8 | QQSNSWPLT |
| | 3 | | |
| 13B3- | Full- | 24 | EIVLTQSPATLSLSPGERATLSCRASQSVSNSLHWYQQKP |
| huVL4 | length | | GQAPRLLIMYTSQSISGIPARFSGSGSGTDFTLTISSLEPED |
| | | | FAVYYCQQSNSWPLTFGGGTKLELK |
| | CDR | 6 | RASQSVSNSLH |
| | 1 | | |
| | CDR | 7 | YTSQSIS |
| | 2 | | |
| | CDR | 8 | QQSNSWPLT |
| | 3 | | |

The humanized VH and VL described above were paired to obtain the corresponding humanized antibodies:

TABLE 9
Exemplary different combinations of light and heavy chains
of humanized antibodies and their corresponding numbers:

| Source of Humanized | Light/Heavy Chain Combination | Combination |
|---|---|---|
| Antibody | of Humanized Antibody | Number |
| 13B3 | huVH4 + huVL1 | P33425 |
| 13B3 | huVH2 + huVL2 | P33426 |
| 13B3 | huVH2 + huVL3 | P33430 |
| 13B3 | huVH2 + huVL4 | P33433 |
| 13B3 | huVH3 + huVL4 | P33434 |

### Example 9 Binding of Humanized Antibodies to Antigen CD27

### 9.1 Study on the In Vitro Binding Activity of Humanized Monoclonal CD27 Antibodies To the CD27 Antigen Using ELISA

To determine whether the humanized CD27 monoclonal antibodies of the present disclosure can bind to the CD27 antigen, an in vitro test was performed, and the CD27 monoclonal antibody Varlilumab (1F5) was used as a positive control.

The test method is the same as that of Example 3.1, and the results are shown in FIG. 10 and Table 10.

TABLE 10

EC50

| | | |
|---|---|---|
| 1F5 | 0.0603 | 0.404 |
| 13B3-hFc | 0.0503 | 0.337 |
| 13B3-huH4L1(P33425) | 0.0371 | 0.249 |
| 13B3-huH2L2(P33426) | 0.0486 | 0.325 |
| 13B3-huH2L3(P33430) | 0.0418 | 0.280 |
| 13B3-huH2L4(P33433) | 0.0409 | 0.274 |
| 13B3-huH3L4(P33434) | 0.0361 | 0.242 |
| | ug/mL | nM |

### Experimental Results and Analysis:

Five humanized monoclonal antibodies (13B3-huH4L1 (P33425), 13B3-huH2L2 (P33426), 13B3-huH2L3 (P33430), 13B3-huH2L4 (P33433), and 13B3-huH3L4 (P33434)) effectively bound to solid-phase CD27, with EC50 values of 0.249 nM, 0.325 nM, 0.280 nM, 0.274 nM, and 0.242 nM, respectively. The EC50 values for the chimeric antibody 13B3-hFc and the positive control 1F5 were 0.337 nM and 0.404 nM, respectively. The humanized antibodies demonstrated superior binding affinity compared to 1F5.

### 9.2 Flow Cytometry to Study the Binding of Humanized CD27 Monoclonal Antibody to CD27 Protein on Cell Membrane

In order to determine whether the humanized CD27 monoclonal antibody of the present invention can bind to the CD27 protein on the CHO cell membrane, an in vitro test was performed, using 1F5 as a positive control.

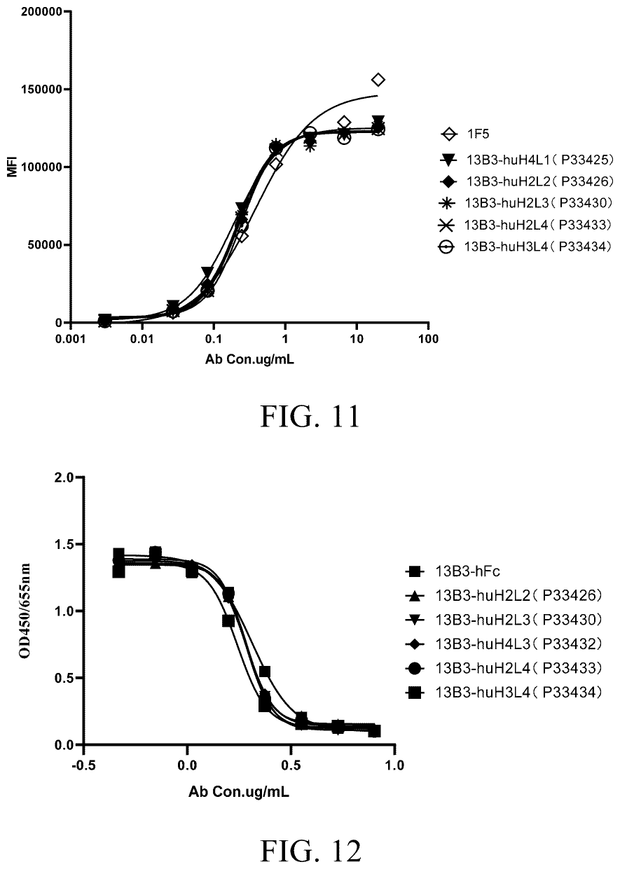

The test method is the same as that of Example 3.2, and the results are shown in FIG. 11 and Table 11.

TABLE 11

EC50

| | | |
|---|---|---|
| 1F5 | 0.386 | 2.584 |
| 13B3-huH4L1(P33425) | 0.190 | 1.270 |
| 13B3-huH2L2(P33426) | 0.221 | 1.478 |
| 13B3-huH2L3(P33430) | 0.211 | 1.411 |
| 13B3-huH2L4(P33433) | 0.224 | 1.501 |
| 13B3-huH3L4(P33434) | 0.242 | 1.620 |
| | ug/mL | nM |

### Experimental Results and Analysis:

Five humanized monoclonal antibodies (13B3-huH4L1 (P33425), 13B3-huH2L2 (P33426), 13B3-huH2L3 (P33430), 13B3-huH2L4 (P33433), and 13B3-huH3L4 (P33434)) all demonstrated effective binding to membrane-form CD27, with EC50values of 1.270 nM, 1.478 nM, 1.411 nM, 1.501 nM, and 1.620 nM, respectively. The EC50 value for the positive control 1F5 was 2.584 nM. These results indicate that the humanized antibodies exhibit superior binding affinity compared to the positive control 1F5.

### Example 10. Inhibition of the Binding of CD27 to CD70 by Humanized CD27 Antibodies

To determine whether the humanized CD27 monoclonal antibodies of the present disclosure can inhibit the binding of CD27 to its ligand CD70, an in vitro ELISA test was performed, using 1F5 as a positive control.

The test method is the same as that of Example 3.5, and the results are shown in FIG. 12 and Table 12.

TABLE 12

IC50

| | | |
|---|---|---|
| 13B3-hFc | 2.059 | 13.795 |
| 13B3-huH2L2(P33426) | 1.916 | 12.837 |
| 13B3-huH2L3(P33430) | 1.954 | 13.092 |
| 13B3-huH4L3(P33432) | 1.911 | 12.804 |
| 13B3-huH2L4(P33433) | 1.911 | 12.804 |
| 13B3-huH3L4(P33434) | 1.746 | 11.698 |
| | ug/mL | nM |

### Experimental Results and Analysis:

Five humanized monoclonal antibodies (13B3-huH2L2 (P33426), 13B3-huH2L3 (P33430), 13B3-huH4L3 (P33432), 13B3-huH2L4 (P33433), and 13B3-huH3L4 (P33434)) effectively inhibited the binding between CD70 and CD27, with IC50 values of 12.837 nM, 13.092 nM, 12.804 nM, 12.804 nM, and 11.698 nM, respectively. The IC50 value for the chimeric antibody 13B3-hFc was 13.795 nM.

### Example 11 Affinity Study of the Binding of Humanized Antibodies to the Antigen Cd27 (SPR Method)

Instrument: Biacore T200 (Cytiva), Probe: CM5 (Lot No.; 10313498).

The ligand was fixed to the probe: After the ligand is prepared, the flow rate was set to 10 uL/min and the ligand was fixed to the probe.

The kinetic parameters set up: the test sample was adjusted to a saturated concentration with 1×HBS-P+ buffer, then diluted to at least 5 concentrations, the flow rate was set to 30 μL/min, and the corresponding binding and dissociation times was set. The reaction temperature defaults to 25° C. for detection.

TABLE 13

CD27

| Sample ID | KD (M) | ka(1/Ms) | kd(1/s) |
|---|---|---|---|
| 13B3-hFc | 7.81e−10 | 1.22e+06 | 9.54e−04 |
| 13B3-huH4L1(P33425) | 6.99e−10 | 1.93e+06 | 1.35e−03 |
| 13B3-huH2L2(P33426) | 6.43e−10 | 1.65e+06 | 1.06e−03 |
| 13B3-huH2L3(P33430) | 3.91e−10 | 1.75e+06 | 6.84e−04 |

### Experimental Results and Analysis:

The affinity of some humanized antibodies to CD27 is better than that of the chimeric antibody 13B3-hFc.

### Example 12 Detection of T Cell Proliferation by Humanized Antibodies

The day before the experiment, anti-CD3 antibody (Biolegend, 317303) and the test antibody were diluted to a fixed concentration with PBS, and 50 μL was added to each well of the cell culture plate. After gentle mixing, the plate was placed in a 4° C. environment overnight. The next day, the PBS antibody dilution solution was aspirated and the plate was placed in an open clean bench for several minutes to dry the remaining liquid. CD3⁺ T cells (Hycells, donor ID: PAZ022) were collected by centrifugation and labeled with CFSE (Biyuntian, C0051) according to the manufacturer's instructions. The cell density was then adjusted to 0.5×10\hat{ }6/mL with complete culture medium, and 200 μL was added to each well of the 96-well plate. The cells were cultured in a 37° C., 5% CO₂ incubator for 72 hours.

After 72 h, the supernatants were collected for detection of IFNγ, and the cells were harvested for detection of cell proliferation.

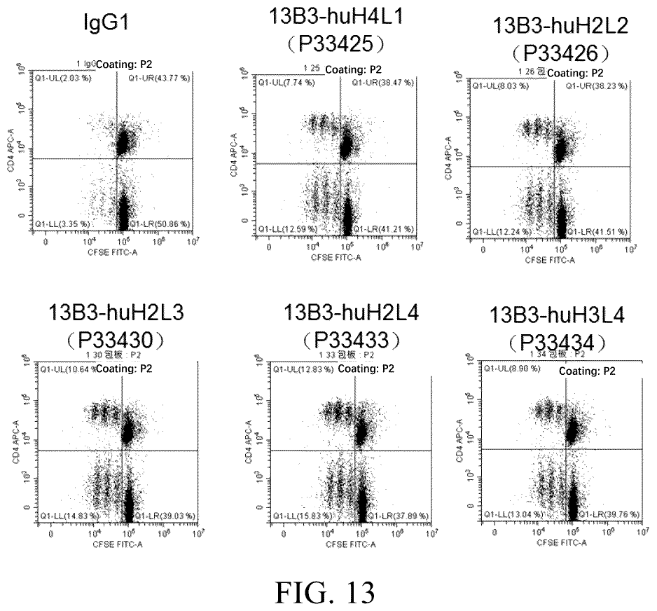

Cell proliferation is shown in FIG. 13;

TABLE 14
IFNγ detection results:

| | IFNγ pg/mL | Fold change (fold) |
|---|---|---|
| IgG1 | 2274 | 1 |
| 13B3-huH4L1(P33425) | 3694 | 1.62 |
| 13B3-huH2L2(P33426) | 4224 | 1.86 |
| 13B3-huH2L3(P33430) | 3454 | 1.52 |
| 13B3-huH2L4(P33433) | 4224 | 1.86 |
| 13B3-huH3L4(P33434) | 3444 | 1.51 |

### Experimental Results and Analysis:

T cell proliferation: Compared to the isotype control IgG1, all anti-CD27 antibodies demonstrated a significant enhancement in the proliferation of CD3+ T cells. Specifically, for the CD3+CD4+ T cell subset, the proliferation rate in the isotype control IgG1 group was 2.03%, while the five humanized antibodies (13B3-huH4L1 (P33425), 13B3-huH2L2 (P33426), 13B3-huH2L3 (P33430), 13B3-huH2L4 (P33433), and 13B3-huH3L4 (P33434)) exhibited proliferation rates of 7.74%, 8.03%, 10.64%, 12.83%, and 8.9%, respectively. For the CD3+CD4− T cell subset, the proliferation rate in the isotype control IgG1 group was 3.35%, whereas the five humanized antibodies showed proliferation rates of 12.59%, 12.24%, 14.83%, 15.83%, and 13.04%, respectively. In summary, all anti-CD27 antibodies demonstrated a significant effect in promoting T cell proliferation.

IFNγ secretion: When T cells receive the first signal from anti-CD3 mAb and the co-stimulatory signal from anti-CD27 mAb, in addition to robust proliferation, they also secrete cytokines including IFNγ. We simultaneously measured the IFNγ levels in the supernatant, observing 1.62-, 1.86-, 1.52-, 1.86-, and 1.51-fold increases for the five humanized antibodies (13B3-huH4L1 (P33425), 13B3-huH2L2 (P33426), 13B3-huH2L3 (P33430), 13B3-huH2L4 (P33433), and 13B3-huH3L4 (P33434)), respectively. In summary, all anti-CD27 antibodies showed a significant effect in promoting IFNγ secretion by T cells.

### Example 13. Effect of Humanized Antibodies on T and B Lymphocyte Levels In Vivo

Human CD27 knock-in (KI) mice were used to study the effects of the humanized antibody 13B3-huH2L4 on T and B lymphocyte levels in vivo.

Twenty-four KI mice were divided into four groups, each consisting of six mice (three females and three males). Each group received intraperitoneal injections of PBS, 13B3-huH2L4 (P33433) at 1 μg/mL and 10 μg/mL, and the positive reference antibody 1F5 at 10 μg/mL, twice weekly for a total of three doses. Blood samples were collected for lymphocyte analysis three and ten days after the final dose.

To 100 μL of blood sample from each mouse, 1 μL each of anti-mouse CD8 APC (BD 553035), anti-mouse CD4 PE (BD 553653), anti-mouse CD3 FITC (BD 555274), and anti-mouse CD19 APC (BioLegend 115512) was added. The mixture was mixed and incubated at 4° C. for 30 minutes. Then, 1 mL of lysis buffer (BD 349202) diluted 10-fold with ddH₂O was added to each sample. The mixture was vortexed for 15 seconds and allowed to stand on ice for 40 minutes to lyse erythrocytes. The cells were centrifuged at 500 g for 5 minutes, the supernatant discarded, and the pellet washed with 1 mL of 0.5% BSA in PBS. The cells were centrifuged again at 500 g for 5 minutes, and the supernatant discarded. Each sample was resuspended in 200 μL of lysis buffer and analyzed by flow cytometry.

### Experimental Results and Analysis:

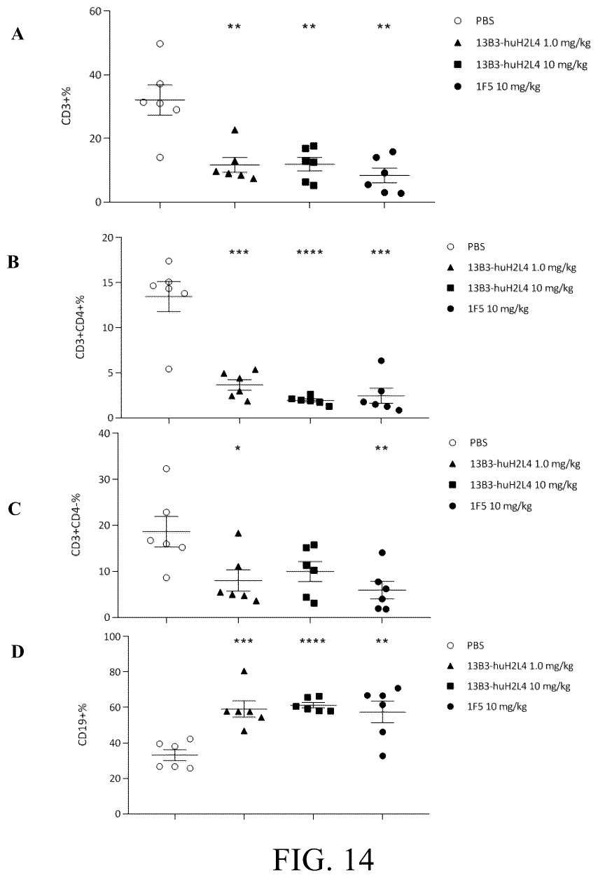

Three days after the last dose, the percentages of CD3+ (FIG. 14A), CD3+CD4+ (FIG. 14B), and CD3+CD4− (FIG. 14C) T lymphocytes in the peripheral blood of the two 13B3-huH2L4 dose groups and the positive control group were significantly decreased compared to the control group, especially CD3+CD4+ T lymphocytes. This suggests that the 13B3-huH2L4 antibody kills CD3+ T lymphocytes with high expression of CD27 on their cell surface. In addition, since B lymphocytes do not have CD27 on their surface, no killing effect on CD19+ B lymphocytes was observed, resulting in a certain increase in the percentage of CD19+ B lymphocytes (FIG. 14D).

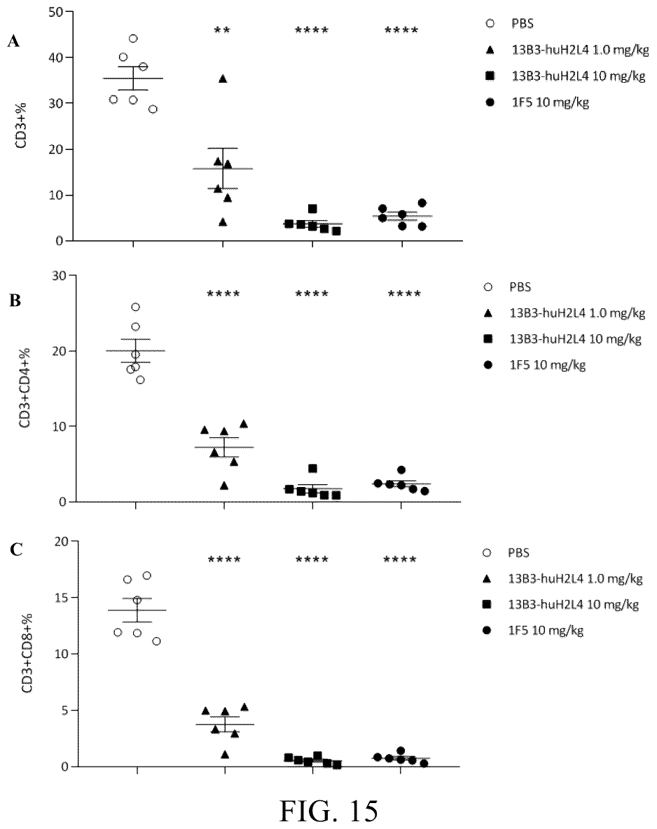

Ten days after the last dose, the proportions of CD3+ (FIG. 15A), CD3+CD4+ (FIG. 15B) and CD3+CD8+ (FIG. 15C) T lymphocytes in the peripheral blood were still significantly lower than those in the PBS control group, and continued to decline compared with 3 days after the last dose.

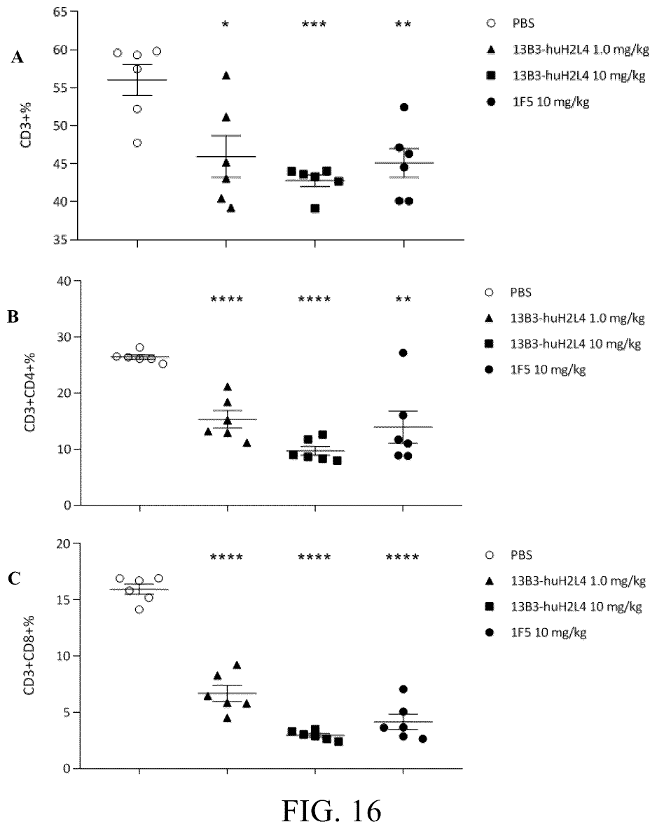

Ten days after the last administration, as in the peripheral blood, the proportions of CD3+ (FIG. 16A), CD3+CD4+ (FIG. 16B), and CD3+CD8+ (FIG. 16C) T lymphocytes in the spleen were significantly lower than those in the PBS control group.

At the same time, the number of various T lymphocytes in peripheral blood was detected, and the changes were consistent with the changes in their percentages.

The results showed that the anti-CD27 antibody of the present invention efficiently kills T lymphocytes that highly express CD27.

### Example 14. Activation of T Lymphocytes after PHA Activation by Humanized Antibodies Combined with Anti-PD-1 or CTLA-4 Antibodies

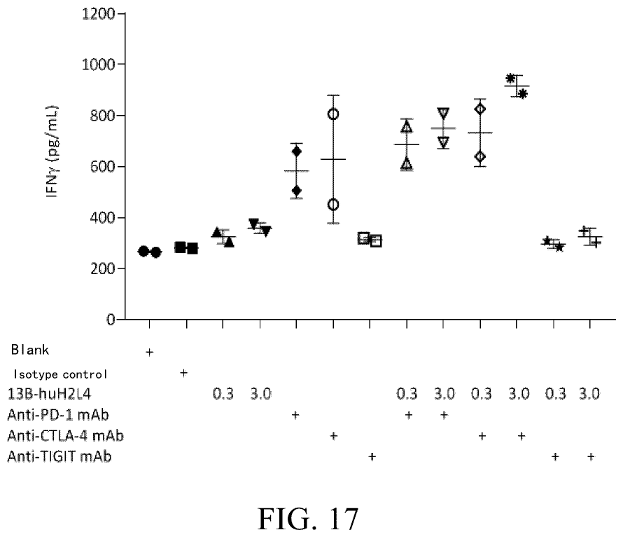

Human PBMCs (TPCS, Lot A19K214031) were seeded into 24-well culture plates and maintained in RPMI 1640 medium supplemented with 10% FBS. T lymphocytes were activated by adding PHA (Sigma, Cat No 9019) to a final concentration of 1 μg/mL. After 3 days of culture, the supernatant was discarded and the cells were washed once. The cells were then transferred to a 96-well culture plate at a density of 5×10⁵ cells per well and cultured in RPMI 1640 medium containing 10% FBS. PHA was added to a final concentration of 1 μg/mL, along with either 13B3-huH2L4 at 0.3 μg/mL or 3 μg/mL, and anti-PD-1, anti-CTLA-4, or anti-TIGIT antibodies at a final concentration of 3 μg/mL (as shown in FIG. 17). The anti-PD-1 antibody used was toripalimab, while the anti-CTLA-4 and anti-TIGIT antibodies were commercially sourced from Celgen Company. After 3 days of culture, supernatants were collected for IFNγ measurement.

### Experimental Results and Analysis:

The experimental results showed (FIG. 17) that 13B3-huH2L4 alone did not enhance the production of IFNγ by exhausted T cells, but anti-PD-1 or CTLA-4 antibodies alone could promote the production of IFNγ by exhausted T cells, and further promoted the production of IFNγ when used in combination with 13B3-huH2L4.

### Example 15. Modification of Humanized CD27 Antibody

15.1 Mutations that Reduce the Ability of Humanized CD27 Antibodies to Bind to Fc Receptors

To reduce antibody-mediated ADCC/ADCP/CDC activity, two amino acids, L234/L235 (EU numbering), in the CH1 region of the heavy chains of humanized antibodies 13B3-huH2L3 and 13B3-huH2L4 were substituted with Ala (LALA variant), thereby abolishing the antibodies' ADCC/ADCP/CDC function. The heavy chain sequences of 13B3-huH2L3 and 13B3-huH2L4 are shown in SEQ ID NO:27, and the heavy chain sequence of the LALA variant is shown in SEQ ID NO:28. The variant genes were generated by artificial gene synthesis. Both variant antibodies, 13B3-huH2L3 (LALA) and 13B3-huH2L4 (LALA), share the LALA variant heavy chain (SEQ ID NO:28).

### 15.2 In Vitro Binding Activity of Variant Antibodies to CD27

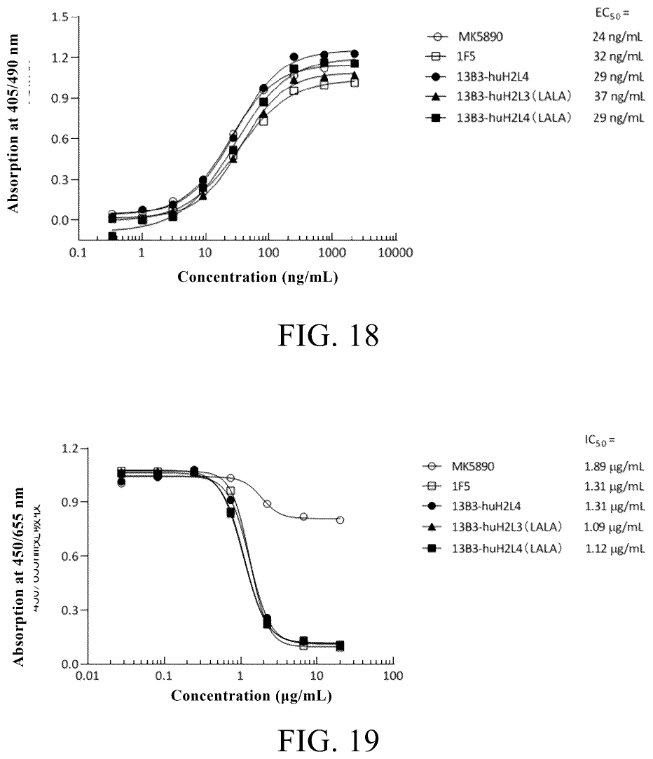

CD27-mFc Coating: CD27 was diluted to a concentration of 1 μg/mL in 1×PBS, mixed thoroughly and added into a 96-well microtiter plate at a 50 μL/well, sealed with film and incubated at 4° C. overnight. Primary Antibody Incubation: serially diluted 13B3-huH2L3 (LALA) and 13B3-huH2L4 (LALA) antibodies were added, as well as various control antibodies (as shown in FIG. 18), to the microtiter plate at 100 μL/well. The plate was sealed with film and incubated at 37° C. for 1 hour. Secondary Antibody Incubation: the blocking buffer was discarded, washed three times with 250 μL of PBST, and patted dry. AP-conjugated anti-human Fc secondary antibody 1:3000 in binding buffer was diluted, and added at 100 μL/well to the microtiter plate, the plate was sealed with film, and incubated at 37° C. for 1 hour. PNPP Incubation: the blocking buffer was discarded, washed three times with 250 μL of PBST, and patted dry. One PNPP pellet was dissolved in 1 mL of 5× diethanolamine substrate, then 4 mL of deionized water were added. 50 μL/well of the solution was added to the plate. The plate was sealed with film and incubated at 37° C. for 15-25 minutes. Detected at dual wavelengths of 405 nm and 490 nm using a microplate reader. The result was calculated as OD405-OD490.

### Experimental Results and Analysis:

The variant antibody 13B3-huH2L4 (LALA) and its parent antibody (wild-type) 13B3-huH2L4 demonstrated comparable binding affinity to human CD27 in vitro, with identical EC50 values of 29 ng/mL. Furthermore, no significant differences in CD27 binding activity were observed between the variant antibody 13B3-huH2L4 (LALA) and the variant antibody 13B3-huH2L3 (LALA), or other control antibodies (1F5 and MK5890) (FIG. 18).

### 15.3. Variant Antibodies Blocking CD70-CD27 Binding Activity

CD70 Coating: CD70 was diluted to a concentration of 1 μg/mL in 1×PBS, mixed thoroughly and added into a 96-well microtiter plate at a concentration of 50 μL/well. The plated was sealed with film and incubated at 4° C. overnight. Plate Blocking: The plate was washed twice with 250 μL/well PBS and patted dry. 150 μL/well of blocking buffer was added, the plate was sealed with film, and incubated at 37° C. for 1.5 hours. Primary Antibody Incubation: the variant 13B3-huH2L3 (LALA) and 13B3-huH2L4 (LALA) antibodies were serially diluted, as well as various control antibodies (as shown in the FIG. ?), in CD27-mFc at a concentration of 100 μL/well. The plate was sealed with film and incubated at 37° C. for 1 hour. The blocking buffer was discarded, and the plate was washed three times with 250 μL PBST, and patted dry. HRP-conjugated anti-mouse Fc secondary antibody was dilute 1:3000 in binding buffer, added to the microplate at 100 μL/well. The plate was sealed with film, and incubated at 37° C. for 1 hour. For TMB incubation: the blocking buffer was discarded, the plate was washed three times with 250 μL of PBST, and patted dry. 50 μL/well of TMB was added to the microplate, the plate was sealed with film, and incubated at 37° C. for 15-25 minutes. The reaction was terminated with HCl (50 μL/well). Detect with a microplate reader at dual wavelengths: 450 nm/655 nm. Calculate the results as OD450-OD655.

### Experimental Results and Analysis:

The variant antibody 13B3-huH2L4 (LALA) showed no substantial difference in its ability to inhibit CD27-CD70 binding in vitro compared to its parent antibody 13B3-huH2L4. Unexpectedly, however, the variant antibody 13B3-huH2L3 (LALA) exhibited moderately enhanced inhibitory activity, with an IC₅₀ of 1.12 μg/mL, whereas the parent antibody 13B3-huH2L4 had an IC₅₀ of 1.31 μg/mL.

Furthermore, the variant antibody 13B3-huH2L3 (LALA) also demonstrated excellent in vitro inhibition of CD27-CD70 binding, with an IC50 of 1.09 μg/mL. This activity was superior to that of the control antibody 1F5 (IC50=1.31 μg/mL) and markedly better than that of the control antibody MK5890 (IC50=1.89 μg/mL) (FIG. 19).

### Example 16. Binding Activity of Variant Antibodies to CD32 and CD64

The binding activity of the variant antibodies to membrane CD32a and membrane CD64 was studied using Jurkat cells stably transfected with the human CD32A gene (Jurkat-CD32a) and CHO-K cells stably transfected with the human CD64 gene (CHO-CD64).

Jurkat-CD32a and CHO-CD64 cells were suspended in 0.5% BSA PBS and added to 96-well round-bottom plates. Each well contained 5×10⁵ 13B3-huH2L4 and 13B3-huH2L4 (LALA) were serially diluted 3-fold and incubated at 4° C. for 1 hour. After washing twice with 0.5% BSA in PBS, 100 μL/well of a 300-fold diluted FITC-labeled anti-human Fc antibody (Jackson ImmunoResearch Lab., #109-116-170) was added and incubated at 4° C. for 0.5 hour. The cells were washed twice with 0.5% BSA in PBS and suspended in 200 μL of PBS buffer for analysis by flow cytometry.

### Experimental Results and Analysis:

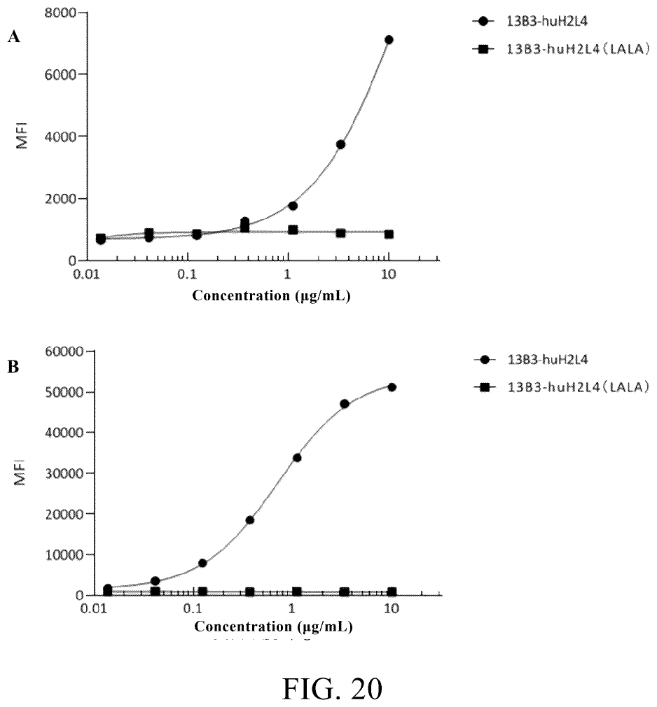

The results showed that the variant antibody 13B3-huH2L4 (LALA) did not bind to CD32a expressed on the cell surface, while its parent wild-type antibody 13B3-huH2L4 did bind normally to CD32a on the cell surface (FIG. 20A). Similarly, the variant antibody 13B3-huH2L4 (LALA) did not bind to CD64 expressed on the cell surface, while its parent wild-type antibody 13B3-huH2L4 did bind normally to CD64 on the cell surface (FIG. 20B).

### Example 17. Effects of Variant Antibodies on T Lymphocytes In Vivo

Human CD27 gene knock-in (KI) mice were used to study the effect of the mutant antibody 13B3-huH2L4 (LALA) on T lymphocyte content in vivo.

Fourteen KI mice were divided into three groups: a PBS control group of two mice and two other groups of four mice each. Each group received 10 mg/kg of 13B3-huH2L4 (LALA) and 10 mg/kg of the positive reference antibody 1F5, respectively. The mice were intraperitoneally administered twice weekly for a total of three doses. Blood samples were collected 3 and 10 days after the final dose.

To 100 μL of blood sample from each mouse, 1 μL each of anti-mouse CD8 APC (BD 553035), anti-mouse CD4 PE (BD 553653), and anti-mouse CD3 FITC (BD 555274) was added. The mixture was mixed and incubated at 4° C. for 30 minutes. Then, 1 mL of lysis buffer (BD 349202) diluted 10-fold in ddH₂O was added to each sample. The samples were vortexed for 15 seconds and allowed to stand on ice for 40 minutes to lyse erythrocytes. The samples were centrifuged at 500 g for 5 minutes, the supernatant discarded, and the pellet washed with 1 mL of 0.5% BSA in PBS. The samples were centrifuged again at 500 g for 5 minutes, and the supernatant discarded. Each sample was resuspended in 200 μL of lysis buffer and analyzed by flow cytometry.

### Experimental Results and Analysis:

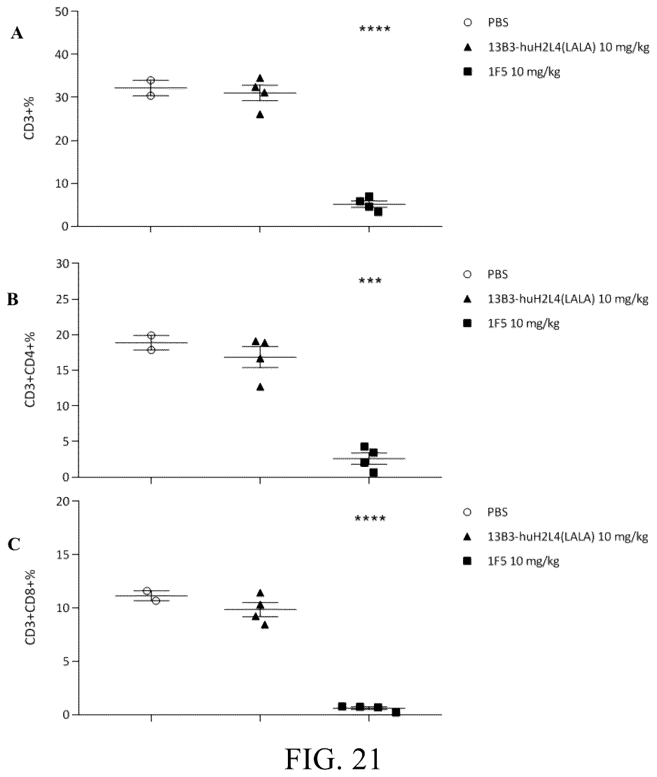

Three days after the final administration, compared with the PBS control group, the positive reference antibody 1F5 significantly reduced the proportions of CD3+ (FIG. 21A), CD3+CD4+ (FIG. 21B), and CD3+CD8+ (FIG. 21C) T lymphocytes in peripheral blood. In contrast, 13B3-huH2L4 (LALA) had no effect on the levels of these T-cell subsets and did not alter their relative distribution in peripheral blood.

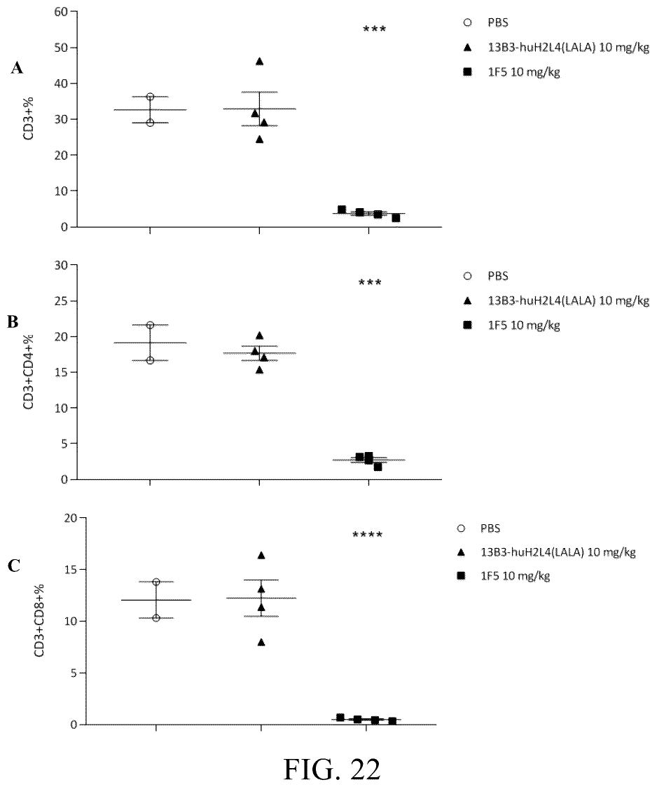

Ten days after the final administration, the proportions of CD3+ (FIG. 22A), CD3+CD4+ (FIG. 22B), and CD3+CD8+ (FIG. 22C) T lymphocytes in the peripheral blood of mice treated with the positive reference antibody 1F5 remained significantly lower than those in the PBS control group, showing no recovery compared to the levels observed three days after dosing. No statistically significant differences were detected between the 13B3-huH2L4 (LALA) group and the PBS control group.

Furthermore, measurements of absolute counts of each T-cell subset in peripheral blood revealed trends consistent with the changes observed in their respective percentages.

These results demonstrate that, unlike the positive reference antibody, the variant antibody 13B3-huH2L4 (LALA) substantially eliminates ADCC activity, thereby exerting no direct cytotoxic effect on CD27-expressing T lymphocytes. The variant antibodies of the present invention, characterized by significantly reduced or abolished ADCC, are particularly suitable for applications requiring T-cell activation while preserving T-cell numbers, such as the treatment of solid tumors and non-T-cell hematological malignancies (e.g., B-cell lymphomas).

All literatures mentioned in the present application are incorporated herein by reference, as though each one is individually incorporated by reference. In addition, it should also be understood that, after reading the above teachings of the present invention, those skilled in the art can make various changes or modifications, equivalents of which falls in the scope of claims as defined in the appended claims.

1. An antibody, wherein the antibody has a heavy chain comprising a heavy chain variable region and a light chain comprising a light chain variable region, wherein the heavy chain variable region has three complementarity determining regions (CDRs) selected from the group consisting of:
(a) CDR1, CDR2 and CDR3 shown in SEQ ID No: 2, 3 and 4;
(b) CDR1, CDR2 and CDR3 shown in SEQ ID No: 2, 25 and 4;
(c) CDR1, CDR2 and CDR3 shown in SEQ ID No: 2, 26 and 4;
(d) CDR1, CDR2 and CDR3 shown in SEQ ID No: 10, 11 and 12;
and the light chain variable region has three complementarity determining regions (L-CDRs) whose sequences are shown in SEQ ID No: 6, 7, and 8;
or the light chain variable region has three complementarity determining regions L-CDRs whose sequences are shown in SEQ ID No: 14, 15, and 16.

2. The antibody of claim 1, wherein the heavy chain variable region comprises the following three complementarity determining regions (CDRs):
(1) complementarity determining region CDR1, wherein the amino acid sequence of the complementarity determining region CDR1 is shown in SEQ ID No: 2;
(2) complementarity determining region CDR2, wherein the amino acid sequence of the complementarity determining region CDR2 is shown in SEQ ID No: 3, 25 or 26; and
(3) complementarity determining region CDR3, the amino acid sequence of the complementary determining region CDR3 is shown in SEQ ID No: 4.

3-8. (canceled)

9. The antibody of claim 1, wherein the Fc of the antibody heavy chain comprises a mutation that causes the loss or substantial loss of ADCC effect of the antibody.

10. The antibody of claim 1, wherein the antibody is selected from the group consisting of:
(Z1) an antibody having a heavy chain variable region shown in SEQ ID No: 1 and a light chain variable region shown in SEQ ID No: 5:
(Z2) an antibody having a heavy chain variable region shown in SEQ ID No: 18 and a light chain variable region shown in SEQ ID No: 22;
(Z3) an antibody having a heavy chain variable region shown in SEQ ID No: 18 and a light chain variable region shown in SEQ ID No: 23;
(Z4) an antibody having a heavy chain variable region shown in SEQ ID No: 18 and a light chain variable region shown in SEQ ID No: 24;
(Z5) an antibody having a heavy chain variable region shown in SEQ ID No: 19 and a light chain variable region shown in SEQ ID No: 24;
(Z6) An antibody having the heavy chain variable region shown in SEQ ID No: 20 and the light chain variable region shown in SEQ ID No: 21.

11. A recombinant protein, wherein the recombinant protein has:
(i) the antibody of claim 1; and
(ii) optionally a tag sequence to facilitate expression and/or purification.

12. An antibody preparation, comprising:
(a) the antibody of claim 1; and
(b) a carrier or excipient.

13. A kit, wherein the kit comprises the antibody of claim 1, and a container for containing the antibody.

14. A CAR construct, wherein the scFv segment of the antigen binding region of the CAR construct is a binding region that specifically binds to CD27, and the scFv has a heavy chain variable region and a light chain variable region, wherein the heavy chain variable region has three complementarity determining regions (CDRs) selected from the group consisting of:
(a) CDR1, CDR2 and CDR3 shown in SEQ ID No: 2, 3 and 4;
(b) CDR1, CDR2 and CDR3 shown in SEQ ID No: 2, 25 and 4;
(c) CDR1, CDR2 and CDR3 shown in SEQ ID No: 2, 26 and 4;
(d) CDR1, CDR2 and CDR3 shown in SEQ ID No: 10, 11 and 12;
and the light chain variable region has three complementarity determining regions (L-CDRs) whose sequences are shown in SEQ ID No: 6, 7, and 8;
or the light chain variable region has three complementarity determining regions L-CDRs whose sequences are shown in SEQ ID No: 14, 15, and 16.

15. A recombinant immune cell, which expresses exogenous CAR construct of claim 14.

16. An antibody-drug conjugate, wherein the antibody-drug conjugate comprises:
(a) an antibody portion which is the antibody of claim 1; and
(b) a conjugated moiety conjugated to the antibody portion, wherein the conjugated moiety is selected from the group consisting of: a detectable label, a drug, a toxin, a cytokine, a radionuclide, an enzyme, or a combination thereof.

17. (canceled)

18. A pharmaceutical composition, comprising:
(i) an active ingredient, the active ingredient is the antibody of claim 1, and
(ii) a pharmaceutically acceptable carrier.

19. A polynucleotide, wherein the polynucleotide encodes
(1) the antibody of claim 1.

20. A method for treating a CD27-related disease, comprising administering to a subject in need thereof the antibody of claim 1, the antibody-drug conjugate of the antibody, or the CAR-T cell expressing the antibody, or a combination thereof.

21. The antibody of claim 1, wherein the light chain variable region comprises the following three complementarity determining regions L-CDR:
(1) complementarity determining region L-CDR1, wherein the amino acid sequence of the complementarity determining region L-CDR1 is shown in SEQ ID No: 6;
(2) complementarity determining region L-CDR2, the amino acid sequence of which is shown in SEQ ID No: 7; and
(3) complementarity determining region L-CDR3, the amino acid sequence of the complementarity determining region L-CDR3 is shown in SEQ ID No: 8.

22. The antibody of claim 1, wherein the antibody has a heavy chain variable region as shown in SEQ ID No: 1, 17, 18, 19 or 20; and/or a light chain variable region as shown in SEQ ID No: 5, 21, 22, 23 or 24.

23. The antibody of claim 1, wherein the antibody has a heavy chain variable region as shown in SEQ ID No: 9; and/or a light chain variable region as shown in SEQ ID No: 13.

24. The antibody of claim 1, wherein the heavy chain sequence is shown in SEQ ID NO: 27, and the constant region of the heavy chain is a wild-type Fc.

25. The antibody of claim 1, wherein the heavy chain sequence is shown in SEQ ID NO: 28, and the constant region of the heavy chain is a mutant Fc, which is the Fc of IgG1 having L234A and L235A mutations.

26. A pharmaceutical composition, comprising:
(i) an active ingredient, the active ingredient is the immune cell of claim 15, and
(ii) a pharmaceutically acceptable carrier.

27. A polynucleotide, wherein the polynucleotide encodes the CAR construct of claim 14.


SEQUENCE LISTING (28 sequences)

SEQ ID NO: 1 - protein, 119 aa, synthetic construct
QVQLQQSGPEQVKPGASVKISCKTSGYAFSQSWMNWVKQRPGQGLEWIGRIYPGDGDTNYNGIFKGKATLTADKSSSTAYMQLSSLTSLDSAVYFCASMIRTTYVMDHWGQGTSVTVTS

SEQ ID NO: 2 - protein, 5 aa, synthetic construct
QSWMN

SEQ ID NO: 3 - protein, 17 aa, synthetic construct
RIYPGDGDTNYNGIFKG

SEQ ID NO: 4 - protein, 10 aa, synthetic construct
MIRTTYVMDH

SEQ ID NO: 5 - protein, 107 aa, synthetic construct
DIVLTQSPATLSVTPGDSVSLSCRASQSVSNSLHWYQQKSHESPRLLIMYTSQSISGIPSRFSGSGSGTDFTLSINSVETEDFGMYFCQQSNSWPLTFGAGTKLELK

SEQ ID NO: 6 - protein, 11 aa, synthetic construct
RASQSVSNSLH

SEQ ID NO: 7 - protein, 7 aa, synthetic construct
YTSQSIS

SEQ ID NO: 8 - protein, 9 aa, synthetic construct
QQSNSWPLT

SEQ ID NO: 9 - protein, 122 aa, synthetic construct
EVKLEESGGGLVQPGGSMKLSCVASGFTFSNYWMNWVRQSPEKGLEWVAEIRLKSNNYATYYAESVKGRFTISRDDSKSSVYLQMNSLRGEDTGIYYCTLYDSSYYYAMDYWGQGTSVTVSS

SEQ ID NO: 10 - protein, 5 aa, synthetic construct
NYWMN

SEQ ID NO: 11 - protein, 19 aa, synthetic construct
EIRLKSNNYATYYAESVKG

SEQ ID NO: 12 - protein, 11 aa, synthetic construct
YDSSYYYAMDY

SEQ ID NO: 13 - protein, 111 aa, synthetic construct
DVVLTQTPLSLPVNIGDQASISCKSTQSLLHSDGFTYLDWYLQKPGQSPQLLIYLVSNRFLGVPDRFSGSGSRTDFTLKISRVEAEDLGVYYCFQSNYLPLTFGGGTKLEI

SEQ ID NO: 14 - protein, 16 aa, synthetic construct
KSTQSLLHSDGFTYLD

SEQ ID NO: 15 - protein, 7 aa, synthetic construct
LVSNRFL

SEQ ID NO: 16 - protein, 9 aa, synthetic construct
FQSNYLPLT

SEQ ID NO: 17 - protein, 119 aa, synthetic construct
QVQLQQSGAEVKKPGASVKISCKTSGYAFSQSWMNWVKQAPGQGLEWIGRIYPGDGDTNYNGIFKGRATLTADKSSSTAYMELSRLRSDDTAVYYCASMIRTTYVMDHWGQGTTVTVSS

SEQ ID NO: 18 - protein, 119 aa, synthetic construct
QVQLVQSGAEVKKPGASVKVSCKTSGYAFSQSWMNWVRQAPGQGLEWIGRIYPGDGDTNYNGIFKGRVTLTADKSSSTAYMELSRLRSDDTAVYYCASMIRTTYVMDHWGQGTTVTVSS

SEQ ID NO: 19 - protein, 119 aa, synthetic construct
QVQLVQSGAEVKKPGASVKVSCKTSGYAFSQSWMNWVRQAPGQGLEWIGRIYPGDGDTNYNGIFQGRVTLTADTSISTAYMELSRLRSDDTAVYYCASMIRTTYVMDHWGQGTTVTVSS

SEQ ID NO: 20 - protein, 119 aa, synthetic construct
QVQLVQSGAEVKKPGASVKVSCKTSGYAFSQSWMNWVRQAPGQGLEWMGRIYPGDGDTNYAQKFQGRVTMTADTSISTAYMELSRLRSDDTAVYYCASMIRTTYVMDHWGQGTTVTVSS

SEQ ID NO: 21 - protein, 107 aa, synthetic construct
EIVLTQSPDFLSVTPKEKVTLTCRASQSVSNSLHWYQQKPDQSPKLLIMYTSQSISGIPSRFSGSGSGTDFTLTINSVEAEDAATYYCQQSNSWPLTFGGGTKLELK

SEQ ID NO: 22 - protein, 106 aa, synthetic construct
IVLTQSPDFQSVTPKEKVTITCRASQSVSNSLHWYQQKPDQSPKLLIMYTSQSISGVPSRFSGSGSGTDFTLTINSLEAEDAATYYCQQSNSWPLTFGGGTKLELK

SEQ ID NO: 23 - protein, 107 aa, synthetic construct
DIVLTQSPATLSVSPGERVTLSCRASQSVSNSLHWYQQKPGQAPRLLIMYTSQSISGIPARFSGSGSGTDFTLTISSVEPEDFAVYYCQQSNSWPLTFGGGTKLELK

SEQ ID NO: 24 - protein, 107 aa, synthetic construct
EIVLTQSPATLSLSPGERATLSCRASQSVSNSLHWYQQKPGQAPRLLIMYTSQSISGIPARFSGSGSGTDFTLTISSLEPEDFAVYYCQQSNSWPLTFGGGTKLELK

SEQ ID NO: 25 - protein, 17 aa, synthetic construct
RIYPGDGDTNYNGIFQG

SEQ ID NO: 26 - protein, 17 aa, synthetic construct
RIYPGDGDTNYAQKFQG

SEQ ID NO: 27 - protein, 448 aa, synthetic construct
QVQLVQSGAEVKKPGASVKVSCKTSGYAFSQSWMNWVRQAPGQGLEWIGRIYPGDGDTNYNGIFKGRVTLTADKSSSTAYMELSRLRSDDTAVYYCASMIRTTYVMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 448 aa, synthetic construct
QVQLVQSGAEVKKPGASVKVSCKTSGYAFSQSWMNWVRQAPGQGLEWIGRIYPGDGDTNYNGIFKGRVTLTADKSSSTAYMELSRLRSDDTAVYYCASMIRTTYVMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG